SEQUENCE LISTING (88 sequences)

SEQ ID NO: 1 - protein, 8 aa, Artificial Sequence
  Synthetic peptide
GGGKGGGG

SEQ ID NO: 2 - protein, 8 aa, Artificial Sequence
  Synthetic peptide
GGGNGSGG

SEQ ID NO: 3 - protein, 8 aa, Artificial Sequence
  Synthetic peptide
GGGCGGGG

SEQ ID NO: 4 - protein, 4 aa, Artificial Sequence
  Synthetic peptide
GPNG

SEQ ID NO: 5 - protein, 22 aa, Artificial Sequence
  Synthetic peptide
QGCSSGGPTLREWQQCVRMQHS

SEQ ID NO: 6 - protein, 22 aa, Artificial Sequence
  Synthetic peptide
QGCSSGGPTLREWQQCRRAQHS

SEQ ID NO: 7 - protein, 22 aa, Artificial Sequence
  Synthetic peptide
QGCSSGGPTLREWQQCVRAQHS

SEQ ID NO: 8 - protein, 18 aa, Artificial Sequence
  Synthetic peptide
CSSGGPTLREWQQCSRAQ

SEQ ID NO: 9 - protein, 18 aa, Artificial Sequence
  Synthetic peptide
CSSGGPTLREWQQCQRAQ

SEQ ID NO: 10 - protein, 18 aa, Artificial Sequence
  Synthetic peptide
CSSGGPTLREWQQCGRAQ

SEQ ID NO: 11 - protein, 22 aa, Artificial Sequence
  Synthetic peptide
QGCSSGGPTLREWQQCVQAQHS

SEQ ID NO: 12 - protein, 22 aa, Artificial Sequence
  Synthetic peptide
QGCSSGGPTLREWQQCVGAQHS

SEQ ID NO: 13 - protein, 22 aa, Artificial Sequence
  Synthetic peptide
QGCSSGGPTLREWQQCVHAQHS

SEQ ID NO: 14 - protein, 22 aa, Artificial Sequence
  Synthetic peptide
QGCSSGGPTLREWQQCQGAQHS

SEQ ID NO: 15 - protein, 22 aa, Artificial Sequence
  Synthetic peptide
QGCSSGGPTLREWQQCVRPQHS

SEQ ID NO: 16 - protein, 22 aa, Artificial Sequence
  Synthetic peptide
QGCSSGGPTLREWQQCFRPQHS

SEQ ID NO: 17 - protein, 22 aa, Artificial Sequence
  Synthetic peptide
QGCSSGGPTLREWQQCFKAQHS

SEQ ID NO: 18 - protein, 22 aa, Artificial Sequence
  Synthetic peptide
QGCSSGGPTLREWQQCVKPQHS

SEQ ID NO: 19 - protein, 22 aa, Artificial Sequence
  Synthetic peptide
QGCSSGGPTLREWQQCVRAQHS

SEQ ID NO: 20 - protein, 22 aa, Artificial Sequence
  Synthetic peptide
QGCSSGGPTLREWQQCRPAQHS

SEQ ID NO: 21 - protein, 22 aa, Artificial Sequence
  Synthetic peptide
QGCSSGGPTLREWQQCRRPQHS

SEQ ID NO: 22 - protein, 22 aa, Artificial Sequence
  Synthetic peptide
QGCSSGGPTLREWQQCQRAQHS

SEQ ID NO: 23 - protein, 22 aa, Artificial Sequence
  Synthetic peptide
QGCSSGGPTLREWQQCSRAQHS

SEQ ID NO: 24 - protein, 228 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 275 aa, Artificial Sequence
  Synthetic polypeptide
AGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 26 - protein, 262 aa, Artificial Sequence
  Synthetic polypeptide
DGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 27 - protein, 253 aa, Artificial Sequence
  Synthetic polypeptide
EGKQVGSGVTTDQVQAEAKESGPTTYKVTSTLTIKEDHRGLTFQQNASSMCVPDQDTAIRVFAIPPSFASIFLTKSTKLTCLVTDLTTYDSVTISWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHDALPNRVTERTVDKSTGKPTLYNVSLVMSDTAGTCY

SEQ ID NO: 28 - protein, 232 aa, Artificial Sequence
  Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 232 aa, Artificial Sequence
  Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 279 aa, Artificial Sequence
  Synthetic polypeptide
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 31 - protein, 228 aa, Artificial Sequence
  Synthetic polypeptide
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 229 aa, Artificial Sequence
  Synthetic polypeptide
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 253 aa, Artificial Sequence
  Synthetic polypeptide
EXKSXDXTVPCPXCPAPELLGGXXXXXXXXXXXXPSVFLFPPKPKDTLMISRTPEVTCVVVDVSHEDPEVXFNWYVDGVEVHNAKTKPREEQFNSTYRVVSVLTVLHQDWLNGKEYKCKVSNKALPAPIEKTISKAKXGQPREPQVYTLPPXSREEMTKNQVSLTCLVKGFYPSDIAVEWESNGQXXPENNYKTTPPMLDSDGXXXSFFLYSKLTVDKSRWQQGNVFSCSVMHEALHNHYTQKSLSLSPGKXX

SEQ ID NO: 34 - protein, 22 aa, Artificial Sequence
  Synthetic peptide
QGCSSGGPTQREWLQCRRMQHS

SEQ ID NO: 35 - protein, 22 aa, Artificial Sequence
  Synthetic peptide
QGCSSGGPTLREWQQCRRMQHS

SEQ ID NO: 36 - protein, 22 aa, Artificial Sequence
  Synthetic peptide
QGCSWGGPTLKIWLQCVRAKHS

SEQ ID NO: 37 - protein, 22 aa, Artificial Sequence
  Synthetic peptide
QGCSWGGPTLKNWLQCVRAKHS

SEQ ID NO: 38 - protein, 22 aa, Artificial Sequence
  Synthetic peptide
QGCSWGGPTLKLWLQCVRAKHS

SEQ ID NO: 39 - protein, 22 aa, Artificial Sequence
  Synthetic peptide
QGCSWGGPTLKHWLQCVRAKHS

SEQ ID NO: 40 - protein, 22 aa, Artificial Sequence
  Synthetic peptide
QGGCRSGPTNREWLACREVQHS

SEQ ID NO: 41 - protein, 22 aa, Artificial Sequence
  Synthetic peptide
QGTCEQGPTLRQWPLCRQGRHS

SEQ ID NO: 42 - protein, 22 aa, Artificial Sequence
  Synthetic peptide
QGTCEQGPTLRLWLLCRQGRHS

SEQ ID NO: 43 - protein, 22 aa, Artificial Sequence
  Synthetic peptide
QGTCEQGPTLRIWLLCRQGRHS

SEQ ID NO: 44 - protein, 254 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCVQAQHSGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 254 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCVGAQHSGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 254 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCVHAQHSGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 254 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCQGAQHSGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 254 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCVRPQHSGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 254 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCFRPQHSGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 254 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCFKAQHSGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 254 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCVKPQHSGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 254 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCVRAQHSGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 254 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCRPAQHSGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 254 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCRRPQHSGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 254 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCQRAQHSGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 254 aa, Artificial Sequence
  Synthetic polypeptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCSRAQHSGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 2 aa, Artificial Sequence
  Synthetic peptide
PP

SEQ ID NO: 58 - protein, 8 aa, Artificial Sequence
  Synthetic peptide
DVSHEDPE

SEQ ID NO: 59 - protein, 4 aa, Artificial Sequence
  Synthetic peptide
VHNA

SEQ ID NO: 60 - protein, 7 aa, Artificial Sequence
  Synthetic peptide
EEQYNST

SEQ ID NO: 61 - protein, 11 aa, Artificial Sequence
  Synthetic peptide
VLHQDWLNGKE

SEQ ID NO: 62 - protein, 16 aa, Artificial Sequence
  Synthetic peptide
NKALPAPIEKTISKAK

SEQ ID NO: 63 - protein, 5 aa, Artificial Sequence
  Synthetic peptide
DELTK

SEQ ID NO: 64 - protein, 7 aa, Artificial Sequence
  Synthetic peptide
NGQPENN

SEQ ID NO: 65 - protein, 11 aa, Artificial Sequence
  Synthetic peptide
TTPPVLDSDGS

SEQ ID NO: 66 - protein, 9 aa, Artificial Sequence
  Synthetic peptide
KSRWQQGNV

SEQ ID NO: 67 - protein, 3 aa, Artificial Sequence
  Synthetic peptide
SHE

SEQ ID NO: 68 - protein, 3 aa, Artificial Sequence
  Synthetic peptide
YNS

SEQ ID NO: 69 - protein, 3 aa, Artificial Sequence
  Synthetic peptide
NKA

SEQ ID NO: 70 - protein, 3 aa, Artificial Sequence
  Synthetic peptide
LTK

SEQ ID NO: 71 - protein, 3 aa, Artificial Sequence
  Synthetic peptide
ENN

SEQ ID NO: 72 - protein, 5 aa, Artificial Sequence
  Synthetic peptide
VLDSD

SEQ ID NO: 73 - protein, 3 aa, Artificial Sequence
  Synthetic peptide
QGN

SEQ ID NO: 74 - protein, 8 aa, Artificial Sequence
  Synthetic peptide
DVSQEDPE

SEQ ID NO: 75 - protein, 7 aa, Artificial Sequence
  Synthetic peptide
EEQFNST

SEQ ID NO: 76 - protein, 11 aa, Artificial Sequence
  Synthetic peptide
VVHQDWLNGKE

SEQ ID NO: 77 - protein, 22 aa, Artificial Sequence
  Synthetic peptide
NKALPAPIEKTISKTKGQPREP

SEQ ID NO: 78 - protein, 22 aa, Artificial Sequence
  Synthetic peptide
NKGLPAPIEKTISKTKGQPREP

SEQ ID NO: 79 - protein, 18 aa, Artificial Sequence
  Synthetic peptide
NKGLPSSIEKAKGQPREP

SEQ ID NO: 80 - protein, 5 aa, Artificial Sequence
  Synthetic peptide
EEMTK

SEQ ID NO: 81 - protein, 7 aa, Artificial Sequence
  Synthetic peptide
SGQPENN

SEQ ID NO: 82 - protein, 11 aa, Artificial Sequence
  Synthetic peptide
TTPPMLDSDGS

SEQ ID NO: 83 - protein, 9 aa, Artificial Sequence
  Synthetic peptide
KSRWQQGNI

SEQ ID NO: 84 - protein, 9 aa, Artificial Sequence
  Synthetic peptide
KSRWQEGNV

SEQ ID NO: 85 - protein, 183 aa, Artificial Sequence
  Synthetic polypeptide
EGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSSRWQQGNVFSCSVMHEALHNHYTQKSLSLSPGK

SEQ ID NO: 86 - protein, 205 aa, Artificial Sequence
  Synthetic polypeptide
SVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGTPRGPQVYTMAPPKEEMTQSQVSITCMVKGFYPPDIYTEWKMNGQPQENYKNTPPTMDTDGSYFLYSKLNVKKETWQQGNTFTCSVLHEGLHNHHTEKSLSH

SEQ ID NO: 87 - protein, 204 aa, Artificial Sequence
  Synthetic polypeptide
SVFIFPPKXKDXLXISXTPXVTCVVVDISXXDPEVKFXWFIDXVEVHXAXTXXXEXQXNSTXRXVSXLILHXDWLNGKXFKCKVXXXAXPAPIEKSISKXXGXPRXPQVYTLXPXKDELTXXQVSITCLVKGFYPXDIXXEWXXNGQPXXNYKXTPPXLDSDGSFFLYSKLXVXKXXWQQGNXFSCSVLHEALHNHHTXKSLSX

SEQ ID NO: 88 - protein, 242 aa, Artificial Sequence
  Synthetic peptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGQGCSSGGPTLREWQQCRRAQHSGGTKNQVSLTCLVKGFYPSDIAVEWESNGQPENNYKTTPPVLDSDGSFFLYSKLTVDKSRWQQGNVFSCSVMHEALHNH

(19) United States
(12) Patent Application Publication
(10) Pub. No.: US 2012/0253009 A1
(43) Pub. Date: Oct. 4, 2012

(54) THROMBOPOIETIC COMPOUNDS

(75) Inventor: Kenneth W. Walker, Newbury Park, CA (US)

(73) Assignee: AMGEN INC., Thousand Oaks, CA (US)

(21) Appl. No.: 13/502,107

(22) PCT Filed: Oct. 14, 2010
(86) PCT No.: PCT/US2010/052722

§ 371 Date: Jun. 20, 2012

Related U.S. Application Data
(60) Provisional application No. 61/252,599, filed on Oct. 16, 2009.

(51) Int. Cl. C07K 19/00; C12N 1/21; C12P 21/02; C12N 5/10; C12N 15/62; C12N 15/63

(52) U.S. Cl. 530/324; 530/350; 530/387.3; 536/23.4; 435/320.1; 435/69.6; 435/69.7; 435/325; 435/358; 435/352; 435/365; 435/328; 435/252.33

(57) ABSTRACT

The invention relates to the field of compounds, especially peptides or polypeptides, that have thrombopoietic activity. The peptides and polypeptides of the invention may be used to increase platelets or platelet precursors (e.g., megakaryocytes) in a mammal.

### RELATED APPLICATIONS

This application is the National Stage of International Application No. PCT/US2010/052722, filed Oct. 14, 2010, which claims the benefit of U.S. Provisional Application No. 61/252,599, filed on Oct. 16, 2009, the contents of which are hereby incorporated by reference in their entirety.

### FIELD OF THE INVENTION

Generally, the invention relates to the field of compounds, especially peptides and polypeptides that have thrombopoietic activity. The compounds of the invention may be used to increase of production platelets or platelet precursors (e.g., megakaryocytes) in a mammal.

### BACKGROUND OF THE INVENTION

The cloning of endogenous thrombopoietin (TPO) (Lok et al., Nature 369:568-571 (1994); Bartley et al., Cell 77:1117-1124 (1994); Kuter et al., Proc. Natl. Acad. Sci. USA 91:11104-11108 (1994); de Sauvage et al., Nature 369:533-538 (1994); Kato et al., Journal of Biochemistry 119:229-236 (1995); Chang et al., Journal of Biological Chemistry 270:511-514 (1995)) has rapidly increased our understanding of megakaryopoiesis (megakaryocyte production) and thrombopoiesis (platelet production).

Endogenous human TPO, a 60 to 70 kDa glycosylated protein primarily produced in the liver and kidney, consists of 332 amino acids (Bartley et al., Cell 77:1117-1124 (1994); Chang et al., Journal of Biological Chemistry 270:511-514 (1995)). The protein is highly conserved between different species, and has 23% homology with human erythropoietin (Gurney et al., Blood 85:981-988 (1995)) in the amino terminus (amino acids 1 to 172) (Bartley et al., Cell 77:1117-1124 (1994)). Endogenous TPO has been shown to possess all of the characteristics of the key biological regulator of thrombopoiesis. Its in vitro actions include specific induction of megakaryocyte colonies from both purified murine hematopoietic stem cells (Zeigler et al., Blood 84:4045-4052 (1994)) and human CD34⁺ cells (Lok et al., Nature 369:568-571 (1994); Rasko et al., Stem Cells 15:33-42 (1997)), the generation of megakaryocytes with increased ploidy (Broudy et al., Blood 85:402-413 (1995)), and the induction of terminal megakaryocyte maturation and platelet production (Zeigler et al., Blood 84:4045-4052 (1994); Choi et al., Blood 85:402-413 (1995)). Conversely, synthetic antisense oligodeoxynucleotides to the TPO receptor (c-Mpl) significantly inhibit the colony-forming ability of megakaryocyte progenitors (Methia et al., Blood 82:1395-1401 (1993)). Moreover, c-Mpl knock-out mice are severely thrombocytopenic and deficient in megakaryocytes (Alexander et al., Blood 87:2162-2170 (1996)).

In general, the interaction of a protein ligand with its receptor often takes place at a relatively large interface. However, as demonstrated in the case of human growth hormone bound to its receptor, only a few key residues at the interface actually contribute to most of the binding energy (Clackson, T. et al., Science 267:383-386 (1995)). This and the fact that the bulk of the remaining protein ligand serves only to display the binding epitopes in the right topology makes it possible to find active ligands of much smaller size.

In an effort toward this, the phage peptide library display system has emerged as a powerful technique in identifying small peptide mimetics of large protein ligands (Scott, J. K. et al., Science 249:386 (1990); Devlin, J. J. et al., Science 249:404 (1990)).

Further, in an effort to seek small structures as lead compounds in the development of therapeutic agents with more desirable properties, a different type of dimer of TMP and related structures were designed in which the C-terminus of one TMP peptide was linked to the N-terminus of a second TMP peptide, either directly or via a linker and the effects of this dimerization strategy on the bioactivity of the resulting dimeric molecules were then investigated (U.S. Pat. No. 6,835,809, Liu et al.; incorporated herein by reference in its entirety). In some cases, these so-called tandem dimers (C-N link) were designed to have linkers between the two monomers, the linkers being preferably composed of natural amino acids, therefore rendering their synthesis accessible to recombinant technologies (U.S. Pat. No. 6,835,809, supra). In addition, the tandem dimers may be further attached to one or more moieties that are derived from immunoglobulin proteins, referred to generally as the Fc region of such immunoglobulins. The resulting compounds are referred to as Fc fusions of TMP tandem dimers (U.S. Pat. No. 6,835,809, supra).

Structural analysis of protein-protein interaction may also be used to suggest peptides that mimic the binding activity of large protein ligands. In such an analysis, the crystal structure may suggest the identity and relative orientation of critical residues of the large protein ligand, from which a peptide may be designed. See, e.g., Takasaki et al., Nature Biotech. 15: 1266-1270 (1997). Hereinafter, these and related methods are referred to as “protein structural analysis.” These analytical methods may also be used to investigate the interaction between a receptor protein and peptides selected by phage display, which may suggest further modification of the peptides to increase binding affinity.

The art would benefit from further technology enabling such rational design of polypeptide therapeutic agents, because there remains a need in the art for additional compounds that have a biological activity of stimulating the production of platelets (thrombopoietic activity) and/or platelet precursor cells, especially megakaryocytes (megakaryopoietic activity).

### SUMMARY OF THE INVENTION

Provided herein is a group of compounds that are capable of binding to and triggering a transmembrane signal through, i.e., activating, the c-Mpl receptor, which is the same receptor that mediates the activity of endogenous thrombopoietin (TPO). Thus, the compounds have thrombopoietic activity, i.e., the ability to stimulate, in vivo and in vitro, the production of platelets, and/or megakaryocytopoietic activity, i.e., the ability to stimulate, in vivo and in vitro, the production of platelet precursors.

The compounds comprise polypeptides or peptides modified to include at least one antibody Fc region and, optionally, one or more water soluble polymers.

In one aspect, a substantially homogenous compound is provided comprising a structure set out in Formula I,

[(X¹)ₐ-(F¹)_{z}-(X²)_{b}]-(L¹)_{c}-WSP_{d} Formula I

and multimers thereof, wherein:
F¹ is a vehicle;

X¹ is independently selected from:
P¹-(L²)ₑ-
P2-(L³)_{f}-P¹-(L²)ₑ-
P³-(L⁴)_{g}-P²-(L³)_{f}-P¹-(L²)ₑ- and
P⁴-(L⁵)ₕ-P³-(L⁴)_{g}-P²-(L³)_{f}-P¹-(L²)ₑ-
   X² is independently selected from:
(L²)ₑ-P¹,
(L²)ₑ-P¹-(L³)_{f}-P²,
(L²)ₑ-P¹-(L³)_{f}-P²-(L⁴)_{g}-P³, and
(L²)ₑ-P¹-(L³)_{f}-P²-(L⁴)_{g}-P³-(L⁵)ₕ-P⁴
   wherein P¹, P², P³, and P⁴ are each independently sequences of pharmacologically active peptides;

L¹, L², L³, L⁴, and L⁵ are each independently linkers;

a, b, c, d, e, f, g, and h are each independently 0 or 1;

z is 0, 1, 2, or more; and
WSP is a water soluble polymer, the attachment of which is effected at any reactive moiety in F¹; said compound having a property of improved bioefficacy when administered in a multidose regimen. In one aspect, the compound is a multimer, and in another aspect, the compound is a dimer.

In one embodiment, the invention provides a compound of Formula I comprising a structure set out in Formula II

[X¹-(F¹)_{z}]-(L¹)_{c}-WSP_{d} Formula II

wherein F¹ is an Fc domain and is attached at the C-terminus of X¹, and zero, one, or more WSP is attached to the Fc domain, optionally through linker L¹. Compounds having this structure are provided as a multimer in one aspect and a dimer in another aspect.

In another embodiment, the invention provides a compound of Formula I comprising a structure set out in Formula III

[(F¹)_{z}-X²]-(L¹)_{c}-WSP_{d} Formula III

wherein F¹ is an Fc domain and is attached at the N-terminus of X², and zero, one, or more WSP is attached to the Fc domain, optionally through linker L¹. Multimers and dimers of a compound having this structure are also provided.

The invention also provides a compound of Formula I comprising a structure set out in Formula IV

[(F¹)_{z}-(L¹)ₑ-P¹]-(L¹)_{c}-WSP_{d} Formula IV

wherein F¹ is an Fc domain and is attached at the N-terminus of -(L¹)_{c}-P¹ and, zero, one, or more WSP is attached to the Fc domain, optionally through linker L¹. Multimers and dimers of a compound having this structure are also provided.

The invention further contemplates a compound of Formula I comprising a structure set out in Formula V

[(F¹)_{z}-(L¹)ₑ-P¹-(L²)_{f}-P²](L¹)_{c}-WSP_{d} Formula V

wherein F¹ is an Fc domain and is attached at the N-terminus of -L¹-P¹-L²-P² and, zero, one, or more WSP is attached to the Fc domain, optionally through linker L¹. Multimers and dimers of a compound having this structure are also provided.

In one aspect, a compound is provided as described above wherein P¹ and/or P² are independently selected from a TPO mimetic set out in any of Tables 1-3,5,7,8, and 11 (see Examples herein). In one aspect, P¹ and/or P² have the same amino acid sequence.

In another aspect, a compound is provided as described above wherein L₁ is a linker group which is optional and, if present, is independently selected from the linker groups consisting of

Yₙ, wherein Y is a naturally-occurring amino acid or a stereoisomer thereof and n is 1 through 20;

(Gly)ₙ, wherein n is 1 through 20, and when n is greater than 1, up to half of the Gly residues may be substituted by another amino acid selected from the remaining 19 natural amino acids or a stereoisomer thereof;

(Gly)₃-Lys(Gly)₄ (SEQ ID NO: 1);

(Gly)₃AsnGlySer(Gly)₂ (SEQ ID NO: 2);

(Gly)₃Cys(Gly)₄ (SEQ ID NO: 3);

GlyProAsnGly (SEQ ID NO: 4);

a Cys residue; and

(CH₂)ₙ, wherein n is 1 through 20.

In one aspect, L is selected from the group consisting of Yₙ, wherein Y is selected a naturally-occurring amino acid or a stereoisomer thereof and n is 1 through 20. In another aspect, L comprises (Gly)ₙ, wherein n is 1 through 20, and when n is greater than 1, up to half of the Gly residues may be substituted by another amino acid selected from the remaining 19 natural amino acids or a stereoisomer thereof. In yet another aspect, L is selected from the group consisting of

(SEQ ID NO: 1)
(Gly)₃Lys(Gly)₄;
(SEQ ID NO: 2)
(Gly)₃AsnGlySer(Gly)₂;
(SEQ ID NO: 3)
(Gly)₃Cys(Gly)₄;
and
(SEQ ID NO: 4)
GlyProAsnGly.

In a further aspect of the invention, L comprises a Cys residue. In another aspect, the invention includes a compound wherein L comprises (CH₂)ₙ, wherein n is 1 through 20.

In another aspect, a compound of the invention is provided as described herein wherein F¹ is an Fc domain. In another aspect, a compound is provided wherein WSP is PEG. In yet another aspect, a compound as described above is provided wherein F¹ is an Fc domain and WSP is PEG.

In one aspect, the PEG component of a compound described herein has a molecular weight of between about 2 kDa and 100 kDa. In another aspect, the PEG component of a compound described herein has a molecular weight of between about 6 kDa and 25 kDa.

The invention further provides a composition comprising a compound described herein wherein the composition comprises at least 50% PEGylated compound. In another aspect, the composition comprises at least 75% PEGylated compound, at least 85% PEGylated compound, at least 90% PEGylated compound, at least 95% PEGylated compound, and at least 99% PEGylated compound.

The invention also provides a method of treating a hematopoietic disorder comprising administering a compound or composition described herein in a regimen effective to treat said disorder.

In one embodiment, the invention includes a compound of a structure set out in Formula I wherein at least a or b is 1.

In another embodiment, the invention includes a compound of a structure set out in Formula I wherein b, c, d, e, f, g, and h are 0.

In a further embodiment, the invention includes a compound that binds to an mpl receptor consisting essentially of a structure set out in Formula I.

In another embodiment, the invention includes a compound of a structure set out in Formula I wherein

F¹ is an Fc domain modified so that it comprises at least one X³ in a loop region;

X³ is independently selected from

-(L⁶)ᵢ-P⁵-(L⁷)ⱼ,

-(L⁶)ᵢ-P⁵-(L⁷)ⱼ-P⁶-(L⁸)ₖ,

-(L⁶)ᵢ-P⁵-(L⁷)ⱼ-P⁶-(L⁸)ₖ-P⁷-(L⁹)ₗ, and

-(L⁶)ᵢ-P⁵-(L⁷)ⱼ-P⁶-(L⁸)ₖ-P⁷-(L²)ₗ-P⁸-(L¹⁰)ₘ;

P⁵, P⁶, P⁷, and P⁸ are each independently sequences of pharmacologically active peptides;

L⁶, L⁷, L⁸, L⁹, and L¹⁰ are each independently linkers;

j, k, l, and m are each independently 0 or 1; and

z is 1, 2, or more.

The invention includes a compound of the aforementioned structure wherein a and b are each 0.

In one embodiment, the invention includes a compound wherein the Fc domain comprises an IgG Fc domain. In one aspect, this IgG Fc domain is an IgG1 Fc domain.

Exemplary compounds of the general structure are shown below. Single letter amino acid abbreviations are used for these peptides.

In yet another embodiment, further exemplary compounds are provided below. Single letter amino acid abbreviations for the peptide are used.

(SEQ ID NO: 5)
QGCSSGGPTLREWQQCVRMQHS;
(SEQ ID NO: 6)
QGCSSGGPTLREWQQCRRAQHS;
(SEQ ID NO: 7)
QGCSSGGPTLREWQQCVRAQHS;
(SEQ ID NO: 8)
CSSGGPTLREWQQCSRAQ;
(SEQ ID NO: 9)
CSSGGPTLREWQQCQRAQ;
(SEQ ID NO: 10)
CSSGGPTLREWQQCGRAQ;
(SEQ ID NO: 11)
QGCSSGGPTLREWQQCVQAQHS (FcL2);
(SEQ ID NO: 12)
QGCSSGGPTLREWQQCVGAQHS (FcL3);
(SEQ ID NO: 13)
QGCSSGGPTLREWQQCVHAQHS (FcL4);
(SEQ ID NO: 14)
QGCSSGGPTLREWQQCQGAQHS (FcL5);
(SEQ ID NO: 15)
QGCSSGGPTLREWQQCVRPQHS (FcL6);
(SEQ ID NO: 16)
QGCSSGGPTLREWQQCFRPQHS (FcL7);
(SEQ ID NO: 17)
QGCSSGGPTLREWQQCFKAQHS (FcL8);
(SEQ ID NO: 18)
QGCSSGGPTLREWQQCVKPQHS (FcL9);
(SEQ ID NO: 19)
QGCSSGGPTLREWQQCVRAQHS (FcL10);
(SEQ ID NO: 20)
QGCSSGGPTLREWQQCRPAQHS (FcL11);
(SEQ ID NO: 21)
QGCSSGGPTLREWQQCRRPQHS (FcL12);
(SEQ ID NO: 22)
QGCSSGGPTLREWQQCQRAQHS (FcL13);
and
(SEQ ID NO: 23)
QGCSSGGPTLREWQQCSRAQHS (FcL14).

In another embodiment, any of the exemplary compounds comprising a TPO-mimetic peptide is optionally fused to either an Fc region or inserted into an Fc-Loop, a modified Fc molecule. Fc-Loops are described herein and in U.S. Patent Application Publication No. US2006/0140934 incorporated herein by reference in its entirety. The invention includes such molecules comprising an Fc domain modified to comprise a peptide as an internal sequence (preferably in a loop region) of the Fc domain. The Fc internal peptide molecules in various embodiments include more than one peptide sequence in tandem in a particular internal region, and they may include further peptides in other internal regions. While the putative loop regions are exemplified, insertions in any other non-terminal domains of the Fc are also considered part of this invention.

The compounds in one aspect are peptides, and they are prepared by standard synthetic methods, by phage library, or by any other methods of preparing peptides. The compounds that encompass non-peptide portions are in various aspects synthesized by standard organic chemistry reactions, in addition to standard peptide chemistry reactions when applicable.

The compounds provided are used for therapeutic or prophylactic purposes by incorporating them with appropriate pharmaceutical carrier materials and administering an effective amount to a subject, such as a human (or other mammal).

Also provided are methods of increasing megakaryocytes or platelets in a patient in need thereof, which comprise administering to said patient an effective amount of the compounds of the invention. In one aspect, the amount is from 1 μg/kg to 100 mg/kg.

The invention further provides pharmaceutical compositions comprising any of the compounds of the invention in admixture with a pharmaceutically acceptable carrier thereof.

In another embodiment, the invention provides polynucleotides that encode the compounds of the invention, vectors that comprise the polynucleotides, and host cells that comprise such vectors.

In a further embodiment, the invention provides methods of producing the compounds of the invention which comprise growing such host cells in a suitable nutrient medium and isolating said compound from said cell or nutrient medium.

Other related aspects are also provided in the instant invention.

### DETAILED DESCRIPTION OF THE EMBODIMENTS

### Definitions

The term “comprising” means that a compound may include additional amino acids on either or both of the N- or C-termini of the given sequence. Of course, these additional amino acids should not significantly interfere with the activity of the compound.

The term “vehicle” refers to a molecule that prevents degradation and/or increases half-life, reduces toxicity, reduces immunogenicity, or increases biological activity of a therapeutic protein. Exemplary vehicles include an Fc domain as well as a linear polymer; a branched-chain polymer (see, for example, U.S. Pat. No. 4,289,872 to Denkenwalter et al., issued Sep. 15, 1981; U.S. Pat. No. 5,229,490 to Tam, issued Jul. 20, 1993; WO 93/21259 by Frechet et al., published 28 Oct. 1993); a lipid; a cholesterol group; a carbohydrate or oligosaccharide; or any natural or synthetic protein, polypeptide or peptide that binds to a salvage receptor. Vehicles are further described hereinafter.

The term “native Fc” refers to molecule or sequence comprising the sequence of a non-antigen-binding fragment resulting from digestion of whole antibody, whether in monomeric or multimeric form. The original immunoglobulin source of the native Fc is in one aspect of human origin and may be any of the immunoglobulins. A native Fc is a monomeric polypeptide that may be linked into dimeric or multimeric forms by covalent association (i.e., disulfide bonds), non-covalent association or a combination of both. The number of intermolecular disulfide bonds between monomeric subunits of native Fc molecules ranges from one to four depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG. Ellison et al. (1982), Nucleic Acids Res. 10: 4071-9. The term “native Fc” as used herein is generic to the monomeric, dimeric, and multimeric forms.

The term “Fc variant” refers to a molecule or sequence that is modified from a native Fc, but preferably still comprises a binding site for the salvage receptor, FcRn. International applications WO 97/34631 (published 25 Sep. 1997) and WO 96/32478 describe exemplary Fc variants, as well as interaction with the salvage receptor, and are hereby incorporated by reference. In one aspect, the term “Fc variant” comprises a molecule or sequence that is humanized from a non-human native Fc. In another aspect, a native Fc comprises sites that may be removed because they provide structural features or biological activity that are not required for the fusion molecules of the present invention. Thus, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, (7) binding to the FcRn salvage receptor in cases where a shorter half-life is desired, or (8) antibody-dependent cellular cytotoxicity (ADCC). Fc variants are described in further detail hereinafter.

The term “Fc domain” encompasses native Fc and Fc variant molecules and sequences as defined above. As with Fc variants and native Fcs, the term “Fc domain” includes molecules in monomeric or multimeric form, whether digested from whole antibody or produced by other means. In one embodiment, for example, the Fc domain or the Fc region can comprise:

(SEQ ID NO: 24)
MDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQV
SLTCLVKGFYPSDIAVEWESNGQPENNYKTTPPVLDSDGSFFLYSKLT
VDKSRWQQGNVFSCSVMEHALHNHYTQKSLSLSPGK.

In another embodiment, other exemplary amino acid sequences (SEQ ID NOS: 25 to 33) of human Fc regions from IgA, IgM and IgG subtypes are also used in the invention.

The term “multimer” as applied to Fc domains or molecules comprising Fc domains refers to molecules having two or more polypeptide chains associated covalently, noncovalently, or by both covalent and non-covalent interactions. IgG molecules typically form dimers; IgM, pentamers; IgD, dimers; and IgA, monomers, dimers, trimers, or tetramers. Multimers may be formed by exploiting the sequence and resulting activity of the native Ig source of the Fc or by derivatizing (as defined below) such a native Fc.

The term “dimer” as applied to Fc domains or molecules comprising Fc domains refers to molecules having two polypeptide chains associated covalently or non-covalently.

The terms “derivatizing,” “derivative” or “derivatized” comprise processes and resulting compounds in which, for example and without limitation, (1) the compound has a cyclic portion; for example, cross-linking between cysteinyl residues within the compound; (2) the compound is cross-linked or has a cross-linking site; for example, the compound has a cysteinyl residue and thus forms cross-linked dimers in culture or in vivo; (3) one or more peptidyl linkage is replaced by a non-peptidyl linkage; (4) the N-terminus is replaced by —NRR₁, NRC(O)R₁, —NRC(O)OR₁, —NRS(O)₂R₁, —NHC(O)NHR, a succinimide group, or substituted or unsubstituted benzyloxycarbonyl-NH—, wherein R and R₁ and the ring substituents are as defined hereinafter; (5) the C-terminus is replaced by —C(O)R₂ or —NR₃R₄ wherein R₂, R₃ and R₄ are as defined hereinafter; and (6) compounds in which individual amino acid moieties are modified through treatment with agents capable of reacting with selected side chains or terminal residues. Derivatives are further described hereinafter.

The term “peptide” refers to molecules of approximately 2 to 80 amino acids, molecules of 2 to 40 amino acids, molecules of 3 to 20 amino acids, and those of 6 to 15 amino acids. For example, peptides having a size selected from no greater than 75, no greater than 70, no greater than 65, no greater than 60, no greater than 55, no greater than 50, no greater than 45, no greater than 40, no greater than 35, no greater than 30, no greater than 25, no greater than 20 amino acids and/or no greater than 15 amino acids, are contemplated herein. Exemplary peptides may be randomly generated by any of the methods cited described herein, carried in a peptide library (e.g., a phage display library), derived by digestion of proteins, or chemically synthesized and the like. Peptides include D and L form, either purified or in a mixture of the two forms. Exemplary peptides are the “biologically active” moieties of the compounds provided herein, i.e., provide the compound with Mpl-binding capacity.

The term “randomized” as used to refer to peptide sequences refers to fully random sequences (e.g., selected by phage display methods) and sequences in which one or more residues of a naturally occurring molecule is replaced by an amino acid residue not appearing in that position in the naturally occurring molecule. Exemplary methods for identifying peptide sequences include phage display, E. coli display, ribosome display, yeast-based screening, RNA-peptide screening, chemical screening, rational design, protein structural analysis, and the like.

The term “pharmacologically active” means that a substance so described is determined to have activity that affects a medical parameter (e.g., blood pressure, blood cell count, cholesterol level) or disease state (e.g., cancer, autoimmune disorders). Thus, pharmacologically active peptides comprise agonistic or mimetic and antagonistic peptides as defined below.

The terms “-mimetic peptide” and “-agonist peptide” refer to a peptide having biological activity comparable to a protein (e.g., TPO) that interacts with a protein of interest. These terms further include peptides that indirectly mimic the activity of a protein of interest, such as by potentiating the effects of the natural ligand of the protein of interest. Those of ordinary skill in the art appreciate that each of these references enables one to select different peptides than actually disclosed therein by following the disclosed procedures with different peptide libraries. Such peptides may mimic the bioactitivy of the large protein ligand or, through competitive binding, inhibit the bioactivity of the large protein ligand, and are commonly referred to as “peptide mimetics” or “mimetic peptides.”

The term “TPO-mimetic peptide” or “TMP” comprises peptides that can be identified or derived as described in International application WO 00/24770, published May 4, 2000, and U.S. Pat. No. 6,835,809, hereby incorporated by reference in their entirety, and any other reference identified as having TPO-mimetic subject matter. Those of ordinary skill in the art appreciate that each of these references enables one to select different peptides than actually disclosed therein by following the disclosed procedures with different peptide libraries.

The term “physiologically acceptable salts” comprises any salts that are known or later discovered to be pharmaceutically acceptable. Some specific examples are: acetate; trifluoroacetate; hydrohalides, such as hydrochloride and hydrobromide; sulfate; citrate; tartrate; glycolate; and oxalate.

The term “W SP” refers to a water soluble polymer which prevents a peptide, protein or other compound to which it is attached from precipitating in an aqueous environment, such as, by way of example, a physiological environment.

The term “PEG” refers to polyethylene glycol, and as used herein is meant to include various forms described in detail infra.

“Substantially homogenous” as used herein with reference to a preparation of the invention means that the preparation includes a single species of a therapeutic compound detectable in the preparation of total therapeutic molecules in the preparation, unless otherwise stated at a specific percentage of total therapeutic molecules. In general, a substantially homogenous preparation is homogenous enough to display the advantages of a homogenous preparation, e.g., ease in clinical application in predictability of lot to lot pharmacokinetics.

“Bioefficacy” refers to the capacity to produce a desired biological effect. Bioefficacy of different compounds, or different dosages of the same compound, or different administrations of the same compound are generally normalized to the amount of compound(s) to permit appropriate comparison.

### Structure of Compounds

Provided herein is a group of compounds that are capable of binding to and triggering a transmembrane signal through, i.e., activating, the c-Mpl receptor, which is the same receptor that mediates the activity of endogenous thrombopoietin (TPO). Thus, the compounds have thrombopoietic activity, i.e., the ability to stimulate, in vivo and in vitro, the production of platelets, and/or megakaryocytopoietic activity, i.e., the ability to stimulate, in vivo and in vitro, the production of platelet precursors.

The compounds comprise polypeptides or peptides modified to include at least one vehicle (i.e., Fc region) attached to the peptide at either the N- or C-terminus and, optionally, one or more WSP covalently attached to the vehicle-peptide molecule at any reactive moiety in the vehicle-peptide molecule.

In one aspect, a substantially homogenous compound is provided comprising a structure set out in Formula I,

[(X¹)ₐ-(F¹)_{z}-(X²)_{b}]-(L¹)_{c}-WSP_{d} Formula I

and multimers thereof, wherein:

F¹ is a vehicle;

X¹ is independently selected from:
P¹-(L²)ₑ-
P²-(L³)_{f}-P¹-(L²)ₑ-
P³-(L⁴)_{g}-P²-(L³)_{f}-P¹-(L²)ₑ- and
P⁴-(L⁵)ₕ-P³-(L⁴)_{g}-P²-(L³)_{f}-P¹-(L²)ₑ-

X² is independently selected from:
-(L²)ₑ-P¹,
-(L²)ₑ-P¹-(L³)_{f}-P²,
-(L²)ₑ-P¹-(L³)_{f}-P²-(L⁴)_{g}-P³, and
-(L²)ₑ-P¹-(L³)_{f}-P²-(L⁴)_{g}-P³-(L⁵)ₕ-P⁴

wherein P¹, P², P³, and P⁴ are each independently sequences of pharmacologically active peptides;

L¹, L², L³, L⁴, and L⁵ are each independently linkers;

a, b, c, d, e, f, g, and h are each independently 0 or 1;

z is 0, 1, 2, or more; and

WSP is a water soluble polymer, the attachment of which is effected at any reactive moiety in F¹;

said compound having a property of improved bioefficacy when administered in a multidose regimen. In one aspect, the compound a multimer, and in another aspect, the compound is a dimer.

The invention also provides a compound of Formula I comprising a structure set out in Formula II

[X¹-(F¹)_{z}]-(L¹)_{c}-WSP_{d} Formula II

wherein F¹ is an Fc domain and is attached at the C-terminus of X¹, and zero, one, or more WSP is attached to the Fc domain, optionally through linker L¹. Compounds having this structure are provided as a multimer in one aspect and a dimer in another aspect.

The invention also provides a compound of Formula I comprising a structure set out in Formula III

[(F¹)_{z}-X²]-(L¹)_{c}-WSP_{d} Formula III

wherein F¹ is an Fc domain and is attached at the N-terminus of X², and zero, one, or more WSP is attached to the Fc domain, optionally through linker L¹. Multimers and dimers of a compound having this structure are also provided.

The invention also provides a compound of Formula I comprising a structure set out in Formula IV

[(F¹)_{z}-(L¹)ₑ-P¹]-(L¹)_{c}-WSP_{d} Formula IV

wherein F¹ is an Fc domain and is attached at the N-terminus of -(L¹)_{c}-P¹ and, zero, one, or more WSP is attached to the Fc domain, optionally through linker L¹. Multimers and dimers of a compound having this structure are also provided.

The invention further provides a compound of Formula I comprising a structure set out in Formula V

[(F¹)_{z}-(L¹)ₑ-P¹-(L²)_{f}-P²]-(L¹)_{c}-WSP_{d} Formula V

wherein F¹ is an Fc domain and is attached at the N-terminus of -L¹-P¹-L²-P² and, zero, one, or more WSP is attached to the Fc domain, optionally through linker L¹. Multimers and dimers of a compound having this structure are also provided.

Provided herein are compounds, as described above, wherein P¹ and/or P² are independently selected from a TPO-mimetic set out in any of Tables 1-3,5,7,8, and 11 herein. In one aspect, P¹ and/or P² have the same amino acid sequence.

In one embodiment, the invention includes a compound of a structure set out in Formula I wherein at least a or b is 1.

In another embodiment, the invention includes a compound of a structure set out in Formula I wherein b, c, d, e, f, g, and h are 0.

In a further embodiment, the invention includes a compound that binds to an mpl receptor consisting essentially of a structure set out in Formula I.

In another embodiment, the invention includes a compound of a structure set out in Formula I wherein

F¹ is an Fc domain modified so that it comprises at least one X³ in a loop region;

X³ is independently selected from
-(L⁶)ᵢ-P⁵-(L⁷)ⱼ,
-(L⁶)ᵢ-P⁵-(L⁷)ⱼ-P⁶-(L⁸)ₖ,

-(L⁶)ᵢ-P⁵-(L⁷)ⱼ-P⁶-(L⁸)ₖ-P⁷-(L⁹)₁, and

-(L⁶)ᵢ-P⁵-(L⁷)ⱼ-P⁶-(L)ₖ-P⁷-(L⁹)ₗ-P⁸-(L¹⁰)ₘ;

P⁵, P⁶, P⁷, and P⁸ are each independently sequences of pharmacologically active peptides;

L⁶, L⁷, L⁸, L⁹, and L¹⁰ are each independently linkers;

j, k, l, and m are each independently 0 or 1; and z is 1, 2, or more.

The invention includes a compound of the aforementioned structure wherein a and b are each 0.

Both three-letter and single letter abbreviations for amino acids are used herein; in each case, the abbreviations are the standard ones used for the 20 naturally-occurring amino acids or well-known variations thereof. These amino acids may have either L or D stereochemistry (except for Gly, which is neither L nor D), and P¹ may comprise a combination of stereochemistries. However, the L stereochemistry is preferred for all of the amino acids in the P¹ chain. The invention also provides reverse P¹ molecules wherein the amino terminal to carboxy terminal sequence of the amino acids is reversed. For example, the reverse of a molecule having the normal sequence Y¹-Y⁷ would be Y⁷—Y¹. The invention also provides retro-reverse P¹ molecules wherein, like a reverse P¹, the amino terminal to carboxy terminal sequence of amino acids is reversed and residues that are normally “L” enantiomers in P¹ are altered to the “D” stereoisomer form.

In addition to the core structure set forth above, Y¹-Y⁷(X₁-X₇), other structures that are specifically contemplated are those in which one or more additional Y groups are attached to the core structure. Thus, one or more Y groups make up the structures U¹ and U². Thus, U¹ and or U² may be attached to the core structure.

Exemplary compounds of the general structure are shown below. Single letter amino acid abbreviations are used for these peptides.

(SEQ ID NO: 34)
QGCSSGGPTQREWLQCRRMQHS
(SEQ ID NO: 35)
QGCSSGGPTLREWQQCRRMQHS
(SEQ ID NO: 36)
QGCSWGGPTLKIWLQCVRAKHS
(SEQ ID NO: 37)
QGCSWGGPTLKNWLQCVRAKHS
(SEQ ID NO: 38)
QGCSWGGPTLKLWLQCVRAKHS
(SEQ ID NO: 39)
QGCSWGGPTLKHWLQCVRAKHS
(SEQ ID NO: 40)
QGGCRSGPTNREWLACREVQHS
(SEQ ID NO: 41)
QGTCEQGPTLRQWPLCRQGRHS
(SEQ ID NO: 42)
QGTCEQGPTLRLWLLCRQGRHS
(SEQ ID NO: 43)
QGTCEQGPTLRIWLLCRQGRHS
(SEQ ID NO: 5)
QGCSSGGPTLREWQQCVRMQHS
(SEQ ID NO: 6)
QGCSSGGPTLREWQQCRRAQHS
(SEQ ID NO: 7)
QGCSSGGPTLREWQQCVRAQHS
(SEQ ID NO: 8)
CSSGGPTLREWQQCSRAQ;
(SEQ ID NO: 9)
CSSGGPTLREWQQCQRAQ;
(SEQ ID NO: 10)
CSSGGPTLREWQQCGRAQ;
(SEQ ID NO: 11)
QGCSSGGPTLREWQQCVQAQHS (FcL2);
(SEQ ID NO: 12)
QGCSSGGPTLREWQQCVGAQHS (FcL3);
(SEQ ID NO: 13)
QGCSSGGPTLREWQQCVHAQHS (FcL4);
(SEQ ID NO: 14)
QGCSSGGPTLREWQQCQGAQHS (FcL5);
(SEQ ID NO: 15)
QGCSSGGPTLREWQQCVRPQHS (FcL6);
(SEQ ID NO: 16)
QGCSSGGPTLREWQQCFRPQHS (FcL7);
(SEQ ID NO: 17)
QGCSSGGPTLREWQQCFKAQHS (FcL8);
(SEQ ID NO: 18)
QGCSSGGPTLREWQQCVKPQHS (FcL9);
(SEQ ID NO: 19)
QGCSSGGPTLREWQQCVRAQHS (FcL10);
(SEQ ID NO: 20)
QGCSSGGPTLREWQQCRPAQHS (FcL11);
(SEQ ID NO: 21)
QGCSSGGPTLREWQQCRRPQHS (FcL12);
(SEQ ID NO: 22)
QGCSSGGPTLREWQQCQRAQHS (FcL13);
and
(SEQ ID NO: 23)
QGCSSGGPTLREWQQCSRAQSH (FcL14).

In addition to the TPO mimetic compounds set forth in Table 11 and as SEQ ID NOs:11-23, also contemplated herein are TPO mimetics comprising various combinations of the mutations introduced to FcL1 (SEQ ID NO:6). For example, contemplated herein is a TPO mimetic peptide having the mutations of FcL10, FcL11, FcL12 introduced into SEQ ID NO:6, such that the amino acids V-P-P occur at positions 17, 18 and 19 relative to SEQ ID NO:6. Another example of a combination contemplated herein is the mutations of FcL13, FcL11, FcL12 introduced into SEQ ID NO:6, such that the amino acids Q-P-P occur at positions 17, 18 and 19 relative to SEQ ID NO:6. Another example of a combination contemplated herein is the mutation of either one of FcL2, FcL3 or FcL4 in combination with FcL12 introduced into SEQ ID NO:6, such that the amino acids V-Q-P (FcL2/Fc12 combo), V-G-P (FcL3/Fc12 combo), or V-H-P (FcL4/Fc12 combo), respectively, occur at positions 17, 18 and 19 relative to SEQ ID NO:6. Another example of a combination contemplated herein is the mutation of FcL8 in combination with FcL12 introduced into SEQ ID NO:6, such that the amino acids F-K-P (FcL8/Fc12 combo) occur at positions 17, 18 and 19 relative to SEQ ID NO:6; and the like.

### Linkers

Any “linker” group (L¹, L², L³, L⁴, and L⁵) is optional. When present, its chemical structure is not critical, since it serves primarily as a spacer. Thus, the terms “linker” and “spacer” may be used interchangeably herein. In one aspect, the linker is made up of amino acids linked together by peptide bonds. Thus, in some embodiments, the linker is made up of from 1 to 20 amino acids linked by peptide bonds, wherein the amino acids are selected from the 20 naturally occurring amino acids. Some of these amino acids may be glycosylated, as is well understood by those in the art. In another embodiment, the 1 to 20 amino acids are selected from glycine, alanine, proline, asparagine, glutamine, and lysine. In a further aspect, a linker is made up of a majority of amino acids that are sterically unhindered, such as glycine and alanine Thus, linkers are polyglycines (particularly (Gly)₄, (Gly)₅), poly(Gly-Ala), and polyalanines. Other specific examples of linkers are:

(SEQ ID NO: 1)
(Gly)₃Lys(Gly)₄;
(SEQ ID NO: 2)
(Gly)₃AsnGlySer(Gly)₂

(this structure provides a site for glycosylation, when it is produced recombinantly in a mammalian cell system that is capable of glycosylating such sites);

(SEQ ID NO: 3)
(Gly)₃Cys(Gly)₄;
and
(SEQ ID NO: 4)
GlyProAsnGly.

To explain the above nomenclature, for example, (Gly)₃Lys(Gly)₄ means Gly-Gly-Gly-Lys-Gly-Gly-Gly-Gly (SEQ ID NO: 1). Combinations of Gly and Ala are also contemplated. The linkers shown here are exemplary; linkers within the scope of this invention may be much longer and may include other residues.

In another embodiment, glycine linkers (or spacers) are used in inserting the TPO-mimetic compounds of the invention into Fc-Loops. These linkers (or spacers) may be symmetric or asymmetric. When linkers (or spacers) are used to connect tandem or multiple peptide sequences, the linkers may be the same or different. Moreover, to the extent where peptides are inserted into other sequences, the linkers at the N- and C-termini may be the same or different.

Non-peptide linkers are also possible. For example, alkyl linkers such as —NH—(CH₂)ₛ—C(O)—, wherein s=2-20 could be used. These alkyl linkers may further be substituted by any non-sterically hindering group such as lower alkyl (e.g., C₁-C₆) lower acyl, halogen (e.g., Cl, Br), CN, NH₂, phenyl, etc. An exemplary non-peptide linker is a PEG linker, which has a molecular weight of 100 to 5000 kD, or 100 to 500 kD. The peptide linkers may be altered to form derivatives as described herein below.

### Derivatives

It is also contemplated that “derivatives” of a TMP (peptide and/or vehicle portion of the TMP) may be substituted for a TMP described above. Such derivatives may improve the solubility, absorption, biological half life, and the like of the compounds. The moieties may alternatively eliminate or attenuate any undesirable side-effect of the compounds and the like.

Such derivative TMPs include compounds in which:

1. The compound or some portion thereof is cyclic. For example, the peptide portion may be modified to contain two or more Cys residues (e.g., in the linker), which could cyclize by disulfide bond formation.

2. The compound is cross-linked or is rendered capable of cross-linking between molecules. For example, the peptide portion may be modified to contain one Cys residue and thereby be able to form an intermolecular disulfide bond with a like molecule. The compound may also be cross-linked through its C-terminus.

3. One or more peptidyl [—C(O)NR—] linkages (bonds) is replaced by a non-peptidyl linkage. Exemplary non-peptidyl linkages are —CH₂-carbamate [—CH2-OC(O)NR—], phosphonate, —CH2-sulfonamide [—CH2-S(O)2NR—], urea [—NHC(O)NH—], —CH2-secondary amine, and alkylated peptide [—C(O)NR6- wherein R6 is lower alkyl].

4. The N-terminus is derivatized. Typically, the N-terminus may be acylated or modified to a substituted amine. Exemplary N-terminal derivative groups include —NRR1 (other than —NH₂), —NRC(O)R1,

—NRC(O)OR1, —NRS(O)₂R1, —NHC(O)NHR1, succinimide, or benzyloxycarbonyl-NH—(CBZ—NH—), wherein R and R1 are each independently hydrogen or lower alkyl with the proviso that R and R1 are not both hydrogen and wherein the phenyl ring may be substituted with 1 to 3 substituents selected from the group consisting of C1-C4 alkyl, C1-C4 alkoxy, chloro, and bromo; to a succinimide group; to a benzyloxycarbonyl-NH—(CBZ—NH—) group; and peptides wherein the free C terminus is derivatized to —C(O)R2 where R2 is selected from the group consisting of lower alkoxy and —NR3R4 where R3 and R4 are independently selected from the group consisting of hydrogen and lower alkyl. By “lower” is meant a group having from 1 to 6 carbon atoms.

5. The free C-terminus is derivatized. Typically, the C-terminus is esterified or amidated. For example, one may use methods described in the art to add (NH—CH2-CH2-NH2)2 to compounds of this invention at the C-terminus. Likewise, one may use methods described in the art to add —NH2 to compounds of this invention at the C-terminus. Exemplary C-terminal derivative groups include, for example, —C(O)R2 wherein R2 is lower alkoxy or —NR3R4 wherein R3 and R4 are independently hydrogen or C1-C8 alkyl (preferably C1-C4 alkyl).

6. A disulfide bond is replaced with another, preferably more stable, cross-linking moiety (e.g., an alkylene). See, e.g., Bhatnagar et al. (1996), J. Med. Chem. 39: 3814-9; Alberts et al. (1993) Thirteenth Am. Pep. Symp., 357-9.

7. One or more individual amino acid residues is modified. Various derivatizing agents are known to react specifically with selected side chains or terminal residues, as described in detail below.

Additionally, modifications of individual amino acids may be introduced into the TMP sequence by reacting targeted amino acid residues of the peptide with an organic derivatizing agent that is capable of reacting with selected side chains or terminal residues. The following are exemplary.

Lysinyl and amino terminal residues may be reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues may be modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine guanidino group.

The specific modification of tyrosyl residues per se has been studied extensively, with particular interest in introducing spectral labels into tyrosyl residues by reaction with aromatic diazonium compounds or tetranitromethane. Most commonly, N-acetyllmidizole and tetranitromethane may be used to form O-acetyl tyrosyl species and 3-nitro derivatives, respectively.

Carboxyl side groups (aspartyl or glutamyl) may be selectively modified by reaction with carbodiimides (R′-N═C═N—R′) such as 1-cyclohexyl-3-(2-morpholinyl-(4-ethyl) carbodiimide or 1-ethyl-3-(4-azonia-4,4-dimethylpentyl) carbodiimide. Furthermore, aspartyl and glutamyl residues may be converted to asparaginyl and glutaminyl residues by reaction with ammonium ions.

Glutaminyl and asparaginyl residues are frequently deamidated to the corresponding glutamyl and aspartyl residues. Alternatively, these residues may be deamidated under mildly acidic conditions. Either form of these residues falls within the scope of this invention.

Cysteinyl residues can be replaced by amino acid residues or other moieties either to eliminate disulfide bonding or, conversely, to stabilize cross-linking See, e.g., Bhatnagar et al. (1996), J. Med. Chem. 39: 3814-9.

Derivatization with bifunctional agents is useful for cross-linking the peptides or their functional derivatives to a water-insoluble support matrix or to other macromolecular carriers. Commonly used cross-linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis (succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 may be employed for protein immobilization.

Other possible modifications include hydroxylation of proline and lysine, phosphorylation of hydroxyl groups of seryl or threonyl residues, oxidation of the sulfur atom in Cys, methylation of the alpha-amino groups of lysine, arginine, and histidine side chains (Creighton, T. E., Proteins: Structure and Molecule Properties, W. H. Freeman & Co., San Francisco, pp. 79-86 (1983)), acetylation of the N-terminal amine, and, in some instances, amidation of the C-terminal carboxyl groups.

Such derivatized moieties preferably improve one or more characteristics including thrombopoietic activity, solubility, absorption, biological half life, and the like of the inventive compounds. Alternatively, derivatized moieties result in compounds that have the same, or essentially the same, characteristics and/or properties of the compound that is not derivatized. The moieties may alternatively eliminate or attenuate any undesirable side effect of the compounds and the like.

As ascertained by peptide mapping and N-terminal sequencing, a preparation is provided which is at least 50% dipolymer/peptide conjugate and at most 50% unreacted peptide and/or monopolymer/peptide conjugate. In other embodiments, preparations are provided which are at least 75% dipolymer/peptide conjugate and at most 25% unreacted peptide and/or monopolymer/peptide conjugate; at least 85% dipolymer/peptide conjugate and at most 15% unreacted peptide and/or monopolymer/peptide conjugate; at least 90% dipolymer/peptide conjugate and at most 10% unreacted peptide and/or monopolymer/peptide conjugate; at least 95% dipolymer/peptide conjugate and at most 5% unreacted peptide and/or monopolymer/peptide conjugate; and at least 99% dipolymerpeptide conjugate and at most 1% unreacted peptide and/or monopolymer/peptide conjugate.

Carbohydrate (oligosaccharide) groups may conveniently be attached to sites that are known to be glycosylation sites in proteins. Generally, O-linked oligosaccharides are attached to serine (Ser) or threonine (Thr) residues while N-linked oligosaccharides are attached to asparagine (Asn) residues when they are part of the sequence Asn-X-Ser/Thr, where X can be any amino acid except proline. X is preferably one of the 19 naturally occurring amino acids other than proline. The structures of N-linked and O-linked oligosaccharides and the sugar residues found in each type are different. One type of sugar that is commonly found on both is N-acetylneuraminic acid (referred to as sialic acid). Sialic acid is usually the terminal residue of both N-linked and O-linked oligosaccharides and, by virtue of its negative charge, may confer acidic properties to the glycosylated compound. Such site(s) may be incorporated in the linker of the compounds of this invention and are preferably glycosylated by a cell during recombinant production of the polypeptide compounds (e.g., in mammalian cells such as CHO, BHK, COS). However, such sites may further be glycosylated by synthetic or semi-synthetic procedures known in the art.

Compounds of the present invention may be changed at the DNA level, as well. The DNA sequence of any portion of the compound may be changed to codons more compatible with the chosen host cell. For E. coli, which is the host cell in one aspect, optimized codons are known in the art. Codons may be substituted to eliminate restriction sites or to include silent restriction sites, which may aid in processing of the DNA in the selected host cell. The vehicle, linker and peptide DNA sequences may be modified to include any of the foregoing sequence changes.

Isotope- and toxin-conjugated derivatives. Another set of useful derivatives are the above-described molecules conjugated to toxins, tracers, or radioisotopes. Such conjugation is especially useful for molecules comprising peptide sequences that bind to tumor cells or pathogens. Such molecules may be used as therapeutic agents or as an aid to surgery (e.g., radioimmunoguided surgery or RIGS) or as diagnostic agents (e.g., radioimmunodiagnostics or RID).

As therapeutic agents, these conjugated derivatives possess a number of advantages. They facilitate use of toxins and radioisotopes that would be toxic if administered without the specific binding provided by the peptide sequence. They also can reduce the side-effects that attend the use of radiation and chemotherapy by facilitating lower effective doses of the conjugation partner.

Useful conjugation partners include:
radioisotopes, such as ⁹⁰Yttrium, ¹³¹Iodine, ²²⁵Actinium, and ²¹³Bismuth;
ricin A toxin, microbially derived toxins such as Pseudomonas endotoxin (e.g., PE38, PE40), and the like;
partner molecules in capture systems (see below);
biotin, streptavidin (useful as either partner molecules in capture systems or as tracers, especially for diagnostic use); and
cytotoxic agents (e.g., doxorubicin).

One useful adaptation of these conjugated derivatives is use in a capture system. In such a system, the molecule of the present invention would comprise a benign capture molecule. This capture molecule would be able to specifically bind to a separate effector molecule comprising, for example, a toxin or radioisotope. Both the vehicle-conjugated molecule and the effector molecule would be administered to the patient. In such a system, the effector molecule would have a short half-life except when bound to the vehicle-conjugated capture molecule, thus minimizing any toxic side-effects. The vehicle-conjugated molecule would have a relatively long half-life but would be benign and non-toxic. The specific binding portions of both molecules can be part of a known specific binding pair (e.g., biotin, streptavidin) or can result from peptide generation methods such as those described herein.

Such conjugated derivatives may be prepared by methods known in the art. In the case of protein effector molecules (e.g., Pseudomonas endotoxin), such molecules can be expressed as fusion proteins from correlative DNA constructs. Radioisotope conjugated derivatives may be prepared, for example, as described for the BEXA antibody (Coulter). Derivatives comprising cytotoxic agents or microbial toxins may be prepared, for example, as described for the BR96 antibody (Bristol-Myers Squibb). Molecules employed in capture systems may be prepared, for example, as described by the patents, patent applications, and publications from NeoRx. Molecules employed for RIGS and RID may be prepared, for example, by the patents, patent applications, and publications from NeoProbe.

The compounds of the invention may also be covalently or noncovalently associated with a carrier molecule, such as a linear polymer (e.g., polyethylene glycol, polylysine, dextran, etc.), a branched-chain polymer (see, for example, U.S. Pat. No. 4,289,872 to Denkenwalter et al., issued Sep. 15, 1981; U.S. Pat. No. 5,229,490 to Tam, issued Jul. 20, 1993; WO 93/21259 by Frechet et al., published 28 Oct. 1993); a lipid; a cholesterol group (such as a steroid); or a carbohydrate or oligosaccharide. Other possible carriers include one or more water soluble polymer attachments such as polyoxyethylene glycol, or polypropylene glycol as described U.S. Pat. Nos. 4,640,835, 4,496,689, 4,301,144, 4,670,417, 4,791,192 and 4,179,337. Still other useful polymers known in the art include monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers.

In one aspect, the carrier is polyethylene glycol (PEG). The PEG group may be of any convenient molecular weight and may be straight chain or branched. The average molecular weight of the PEG will range from about 2 kDa to about 100 kDa, or from about 5 kDa to about 50 kDa, or from about 5 kDa to about 10 kDa.

The PEG groups will generally be attached to the compounds of the invention via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the target compound (e.g., an aldehyde, amino, ester, thiol, α-haloacetyl, maleimido or hydrazino group).

### Vehicles

This invention requires the presence of at least one vehicle (F¹, F²) attached to a peptide through the N-terminus, C-terminus or a side chain of one of the amino acid residues. An Fc domain is a vehicle provided herein. Thus, an Fc domain may be fused to the N or C termini of the peptides or at both the N and C termini. Multiple vehicles may also be used; e.g., Fc's at each terminus or an Fc at a terminus and a PEG group at the other terminus or a side chain.

In various embodiments, the Fc component is either a native Fc or an Fc variant. By way of example and without limitation, the Fc component is preferably the Fc region of the human immunoglobulin IgG1 heavy chain or a biologically active fragment, derivative, or dimer thereof, see Ellison, J. W. et al., Nucleic Acids Res. 10:4071-4079 (1982). Native Fc domains are made up of monomeric polypeptides that may be linked into dimeric or multimeric forms by covalent (i.e., disulfide bonds) and/or non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9).

In one aspect, the Fc sequence shown in SEQ ID NO: 24 is an Fc sequence for the compounds provided herein. Also provided are compounds in which the Fc is a dimeric form of the sequence of SEQ ID NO: 24 and each Fc chain is attached to a TMP tandem dimer. Additional Fc sequences are known in the art and are contemplated for use in the invention. For example, Fc IgG1 (GenBank Accession No. P01857), Fc IgG2 (GenBank Accession No. P01859), Fc IgG3 (GenBank Accession No. P01860), Fc IgG4 (GenBank Accession No. P01861), Fc IgA1 (GenBank Accession No. P01876), Fc IgA2 (GenBank Accession No. P01877), Fc IgD (GenBank Accession No. P01880), Fc IgM (GenBank Accession No. P01871), and Fc IgE (GenBank Accession No. P01854) are some additional Fc sequences contemplated for use herein.

Variants, analogs or derivatives of the Fc portion may be constructed by, for example, making various substitutions of residues or sequences. In one aspect, an Fc variant is incorporated which comprises a molecule or sequence that is humanized from a non-human native Fc. Alternately, an Fc variant comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC), each of which is described in detail in U.S. Patent Application No. 20040087778, the disclosure of which is incorporated by reference in its entirety.

Variant (or analog) polypeptides include insertion variants, wherein one or more amino acid residues supplement an Fc amino acid sequence. Insertions may be located at either or both termini of the protein, or may be positioned within internal regions of the Fc amino acid sequence. Insertion variants, with additional residues at either or both termini, can include for example, fusion proteins and proteins including amino acid tags or labels. For example, the Fc molecule may optionally contain an N-terminal Met, especially when the molecule is expressed recombinantly in a bacterial cell such as E. coli.

In Fc deletion variants, one or more amino acid residues in an Fc polypeptide are removed. Deletions can be effected at one or both termini of the Fc polypeptide, or with removal of one or more residues within the Fc amino acid sequence. Deletion variants, therefore, include all fragments of an Fc polypeptide sequence.

In Fc substitution variants, one or more amino acid residues of an Fc polypeptide are removed and replaced with alternative residues. In one aspect, the substitutions are conservative in nature and conservative substitutions of this type are well known in the art. Alternatively, the invention embraces substitutions that are also non-conservative.

For example, cysteine residues can be deleted or replaced with other amino acids to prevent formation of some or all disulfide crosslinks of the Fc sequences. Each cysteine residue can be removed and/or substituted with other amino acids, such as Ala or Ser. As another example, modifications may also be made to introduce amino acid substitutions to (1) ablate the Fc receptor binding site; (2) ablate the complement (Clq) binding site; and/or to (3) ablate the antibody dependent cell-mediated cytotoxicity (ADCC) site. Such sites are known in the art, and any known substitutions are within the scope of Fc as used herein. For example, see Molecular Immunology, Vol. 29, No. 5, 633-639 (1992) with regard to ADCC sites in IgG1.

Likewise, one or more tyrosine residues can be replaced by phenylalanine residues. In addition, other variant amino acid insertions, deletions and/or substitutions are also contemplated and are within the scope of the present invention. Conservative amino acid substitutions will generally be preferred. Furthermore, alterations may be in the form of altered amino acids, such as peptidomimetics or D-amino acids.

As noted above, both native Fcs and Fc variants are suitable Fc domains for use within the scope of this invention. A native Fc may be extensively modified to form an Fc variant provided binding to the salvage receptor is maintained; see, for example WO 97/34631 and WO 96/32478. In such Fc variants, one may remove one or more sites of a native Fc that provide structural features or functional activity not required by the fusion molecules of this invention. One may remove these sites by, for example, substituting or deleting residues, inserting residues into the site, or truncating portions containing the site. The inserted or substituted residues may also be altered amino acids, such as peptidomimetics or D-amino acids. Fc variants may be desirable for a number of reasons, several of which are described below. Exemplary Fc variants include molecules and sequences in which:

1. Sites involved in disulfide bond formation are removed. Such removal may avoid reaction with other cysteine-containing proteins present in the host cell used to produce the molecules of the invention. For this purpose, the cysteine-containing segment at the N-terminus may be truncated or cysteine residues may be deleted or substituted with other amino acids (e.g., alanyl, seryl). In particular, one may truncate the N-terminal 20-amino acid segment of SEQ ID NO: 24 or delete or substitute the cysteine residues at positions 7 and 10 of SEQ ID NO: 24. Even when cysteine residues are removed, the single chain Fc domains can still form a dimeric Fc domain that is held together non-covalently.

2. A native Fc is modified to make it more compatible with a selected host cell. For example, one may remove the PA sequence near the N-terminus of a typical native Fc, which may be recognized by a digestive enzyme in E. coli such as proline iminopeptidase. One may also add an N-terminal methionine residue, especially when the molecule is expressed recombinantly in a bacterial cell such as E. coli. The Fc domain of SEQ ID NO: 24 is one such Fc variant.

3. A portion of the N-terminus of a native Fc is removed to prevent N-terminal heterogeneity when expressed in a selected host cell. For this purpose, one may delete any of the first 20 amino acid residues at the N-terminus, particularly those at positions 1, 2, 3, 4 and 5.

4. One or more glycosylation sites are removed. Residues that are typically glycosylated (e.g., asparagine) may confer cytolytic response. Such residues may be deleted or substituted with unglycosylated residues (e.g., alanine)

5. Sites involved in interaction with complement, such as the Clq binding site, are removed. For example, one may delete or substitute the EKK sequence of human IgG1. Complement recruitment may not be advantageous for the molecules of this invention and so may be avoided with such an Fc variant.

6. Sites are removed that affect binding to Fc receptors other than a salvage receptor. A native Fc may have sites for interaction with certain white blood cells that are not required for the fusion molecules of the present invention and so may be removed.

7. The ADCC site is removed. ADCC sites are known in the art; see, for example, Molec. Immunol. 29 (5): 633-9 (1992) with regard to ADCC sites in IgG1. These sites, as well, are not required for the fusion molecules of the present invention and so may be removed.

8. When the native Fc is derived from a non-human antibody, the native Fc may be humanized. Typically, to humanize a native Fc, one will substitute selected residues in the non-human native Fc with residues that are normally found in human native Fc. Techniques for antibody humanization are well known in the art.

Preferred Fc variants include the following. In SEQ ID NO: 24, the leucine at position 15 may be substituted with glutamate; the glutamate at position 99, with alanine; and the lysines at positions 101 and 103, with alanines In addition, one or more tyrosine residues can be replaced by phenylalanine residues.

It should be noted that Fc monomers will spontaneously dimerize when the appropriate cysteine residues are present, unless particular conditions are present that prevent dimerization through disulfide bond formation. Even if the cysteine residues that normally form disulfide bonds in the Fc dimer are removed or replaced by other residues, the monomeric chains will generally form a dimer through non-covalent interactions. The term “Fc” herein is used to mean any of these forms: the native monomer, the native dimer (disulfide bond linked), modified dimers (disulfide and/or non-covalently linked), and modified monomers (i.e., derivatives).

Fc sequences may also be derivatized, i.e., bearing modifications other than insertion, deletion, or substitution of amino acid residues. In one aspect, the modifications are covalent in nature, and include for example, chemical bonding with polymers, lipids, other organic, and inorganic moieties. However, non-covalent modifications are also contemplated. Derivatives of the invention may be prepared to increase circulating half-life, or may be designed to improve targeting capacity for the polypeptide to desired cells, tissues, or organs.

It is also possible to use the salvage receptor binding domain of the intact Fc molecule as the Fc part of a compound of the invention, such as described in WO 96/32478, entitled “Altered Polypeptides with Increased Half-Life.” Additional members of the class of molecules designated as Fc herein are those that are described in WO 97/34631, entitled “Immunoglobulin-Like Domains with Increased Half-Lives.” Both of the published PCT applications cited in this paragraph are hereby incorporated by reference.

As discussed herein, the Fc fusions may be at the N or C terminus of a TMP of the invention, or at both the N and C termini of the TMP. It has been previously been shown that peptides in which an Fc moiety is ligated to the N terminus of the TMP group is more bioactive than the other possibilities. When the Fc is fused at the N-terminus of the TMP or linker, such fusion will generally occur at the C-terminus of the Fc chain, and vice versa.

An alternative vehicle would be a protein, polypeptide, peptide, antibody, antibody fragment, or small molecule (e.g., a peptidomimetic compound) capable of binding to a salvage receptor. For example, one could use as a vehicle a polypeptide as described in U.S. Pat. No. 5,739,277, issued Apr. 14, 1998 to Presta et al. Peptides could also be selected by phage display for binding to the FcRn salvage receptor. Such salvage receptor-binding compounds are also included within the meaning of “vehicle” and are within the scope of this invention. Such vehicles should be selected for increased half-life (e.g., by avoiding sequences recognized by proteases) and decreased immunogenicity (e.g., by favoring non-immunogenic sequences, as discovered in antibody humanization).

As noted above, polymer vehicles may also be used for F¹ and F². Various means for attaching chemical moieties useful as vehicles are currently available, see, e.g., Patent Cooperation Treaty (“PCT”) International Publication No. WO 96/11953, entitled “N-Terminally Chemically Modified Protein Compositions and Methods,” herein incorporated by reference in its entirety. This PCT publication discloses, among other things, the selective attachment of water soluble polymers to the N-terminus of proteins.

### Water-Soluble Polymers

This invention contemplates compounds comprising a water-soluble polymer (WSP). Suitable, clinically acceptable, WSP include without limitation, PEG, polyethylene glycol propionaldehyde, copolymers of ethylene glycol/propylene glycol, monomethoxy-polyethylene glycol, carboxymethylcellulose, polyacetals, polyvinyl alcohol (PVA), polyvinyl pyrrolidone, poly-1,3-dioxolane, poly-1,3,6-trioxane, ethylene/maleic anhydride copolymer, poly (.beta.-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and mixtures thereof. In fact, any of the forms of PEG that have been used to derivatize other proteins, such as and without limitation mono-(C1-C10) alkoxy- or aryloxy-polyethylene glycol, are provided. Polyethylene glycol propionaldehyde may have advantages in manufacturing due to its stability in water.

The PEG group may be of any convenient molecular weight and may be linear or branched. The average molecular weight of PEG contemplated for use in the invention ranges from about 2 kDa to about 100 kDa, from about 5 kDa to about 50 kDa, from about 5 kDa to about 10 kDa. In another aspect, the PEG moiety has a molecular weight from about 6 kDa to about 25 kDa. PEG groups generally are attached to peptides or proteins via acylation or reductive alkylation through a reactive group on the PEG moiety (e.g., an aldehyde, amino, thiol, or ester group) to a reactive group on the target peptide or protein (e.g., an aldehyde, amino, or ester group). Using methods described herein, a mixture of polymer/peptide conjugate molecules can be prepared, and the advantage provided herein is the ability to select the proportion of polymer/peptide conjugate to include in the mixture. Thus, if desired, a mixture of peptides with various numbers of polymer moieties attached (i.e., zero, one or two) can be prepared with a predetermined proportion of polymer/protein conjugate.

A useful strategy for the PEGylation of synthetic peptides consists of combining, through forming a conjugate linkage in solution, a peptide and a WSP (PEG) moiety, each bearing a special functionality that is mutually reactive toward the other. The peptides can be easily prepared with conventional solid phase synthesis. The peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The PEGylated peptides can be easily purified by preparative HPLC and characterized by analytical HPLC, amino acid analysis and laser desorption mass spectrometry.

Polysaccharide polymers are another type of WSP which may be used for protein modification. Dextrans are polysaccharide polymers comprised of individual subunits of glucose predominantly linked by α1-6 linkages. The dextran itself is available in many molecular weight ranges, and is readily available in molecular weights from about 1 kD to about 70 kD. Dextran is a suitable water soluble polymer for use in the present invention as a vehicle by itself or in combination with another vehicle (e.g., Fc). See, for example, WO 96/11953 and WO 96/05309. The use of dextran conjugated to therapeutic or diagnostic immunoglobulins has been reported; see, for example, European Patent Publication No. 0 315 456, which is hereby incorporated by reference. Dextran of about 1 kD to about 20 kD is preferred when dextran is used as a vehicle in accordance with the present invention.

The WSP moiety of the molecule may be branched or unbranched. For therapeutic use of the end-product preparation, the polymer is pharmaceutically acceptable. In general, a desired polymer is selected based on such considerations as whether the polymer conjugate will be used therapeutically, and if so, the desired dosage, circulation time, resistance to proteolysis, and other considerations. In various aspects, the average molecular weight of each WSP is between about 2 kDa and about 100 kDa, between about 5 kDa and about 50 kDa, between about 12 kDa and about 40 kDa and between about 20 kDa and about 35 kDa. In yet another aspect the molecular weight of each polymer is between about 6 kDa and about 25 kDa. The term “about” as used herein and throughout, indicates that in preparations of a water soluble polymer, some molecules will weigh more, some less, than the stated molecular weight. Generally, the higher the molecular weight or the more branches, the higher the polymer/protein ratio. Other sizes may be used, depending on the desired therapeutic profile including for example, the duration of sustained release; the effects, if any, on biological activity; the ease in handling; the degree or lack of antigenicity and other known effects of a water soluble polymer on a therapeutic protein.

The WSP should be attached to a peptide or protein with consideration given to effects on functional or antigenic domains of the peptide or protein. In general, chemical derivatization may be performed under any suitable condition used to react a protein with an activated polymer molecule. Activating groups which can be used to link the water soluble polymer to one or more proteins include without limitation sulfone, maleimide, sulfhydryl, thiol, triflate, tresylate, azidirine, oxirane and 5-pyridyl. If attached to the peptide by reductive alkylation, the polymer selected should have a single reactive aldehyde so that the degree of polymerization is controlled.

### Production of Compounds/Methods of Making

The compounds described herein largely may be made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The invention also includes a vector capable of expressing the peptides in an appropriate host. The vector comprises the DNA molecule that codes for the peptides operatively linked to appropriate expression control sequences. Methods of effecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is a preferred technique of making individual peptides since it is the most cost-effective method of making small peptides.

The compounds in one aspect are peptides, and they may be prepared by standard synthetic methods or any other methods of preparing peptides. The compounds that encompass non-peptide portions may be synthesized by standard organic chemistry reactions, in addition to standard peptide chemistry reactions when applicable.

Phage display, in particular, is useful in generating peptides for use in the present invention. It has been stated that affinity selection from libraries of random peptides can be used to identify peptide ligands for any site of any gene product. Dedman et al. (1993), J. Biol. Chem. 268: 23025-30. Phage display is particularly well suited for identifying peptides that bind to such proteins of interest as cell surface receptors or any proteins having linear epitopes. Wilson et al. (1998), Can. J. Microbiol. 44: 313-29; Kay et al. (1998), Drug Disc. Today 3: 370-8. Such proteins are extensively reviewed in Herz et al. (1997), J. Receptor & Signal Transduction Res. 17(5): 671-776, which is hereby incorporated by reference. Such proteins of interest are contemplated for use in this invention.

Peptide compounds are contemplated wherein all of the amino acids have a D configuration, or at least one of the amino acids has a D configuration. It is also contemplated that the peptide compounds may be cyclic.

Compounds that contain derivatized peptides or which contain non-peptide groups may be synthesized by well-known organic chemistry techniques.

A TMP of a preparation of the invention can be prepared using recombinant DNA techniques. Alternatively, a polynucleotide encoding a TMP is prepared using chemical synthesis techniques known in the art, such as the phosphoramidate method. In yet another alternative, a combination of these techniques is used.

### Vectors

For recombinant protein expression, the invention provides a vector encoding a TMP polypeptide which can be expressed in an appropriate host. Such a vector comprises a polynucleotide that encodes a TMP in monomeric or multimer (generally in a tandem structure) arrangement, with or without an Fc domain modification, operatively linked to appropriate expression control sequences. Methods of effecting operative linking, either before or after the DNA molecule is inserted into the vector, are well known in the art. Expression control sequences include promoters, activators, enhancers, operators, ribosomal binding sites, start signals, stop signals, cap signals, polyadenylation signals, and/or other signals involved with the control of transcription or translation. The worker of skill in the art will appreciate that various combinations of these control sequences can be utilized, depending on, for example, the choice of host cell in which the TMP is to be expressed. The resulting vector is transformed into an appropriate host using methods well known in the art.

### Host Cells

Any of a large number of available and well-known host cells is used to express a TMP polypeptide. Selection of a host is dependent upon a number of factors including, for example and without limitation, compatibility with the chosen expression vector, toxicity of the expressed TMP encoded by a transformed polynucleotide, rate of transformation, ease of recovery of the expressed TMP, expression characteristics, degree and type of glycosylation, if desired, bio-safety and costs. A balance of these factors must be struck with the understanding that not all host cells may be equally effective for the expression of a particular TMP. Depending upon the host cell employed, the TMP expression product may be glycosylated with mammalian or other eukaryotic carbohydrates, or it may be non-glycosylated. The TMP expression product may also include an initial methionine amino acid residue (at amino acid residue position-1) if expressed in, for example, a bacterial host cell. Within these general guidelines, useful host cells include bacteria, yeast and other fungi, insects, plants, mammalian (including human) cells in culture, or other host cells known in the art. Host cells are cultured under conventional fermentation conditions well known in the art to permit expression of the desired compounds and the TMP expression product is purified using techniques also known in the art.

Depending on the host cell utilized to express a TMP, carbohydrate (oligosaccharide) groups may conveniently be attached to sites that are known to be glycosylation sites in proteins. Generally, O-linked oligosaccharides are attached to serine (Ser) or threonine (Thr) residues while N-linked oligosaccharides are attached to asparagine (Asn) residues when they are part of the sequence Asn-X-Ser/Thr, where X can be any amino acid except proline. X is preferably one of the 19 naturally occurring amino acids not counting proline. The structures of N-linked and O-linked oligosaccharides and the sugar residues found in each type are different. One type of sugar that is commonly found on both is N-acetylneuraminic acid (referred to as sialic acid). Sialic acid is usually the terminal residue of both N-linked and O-linked oligosaccharides and, by virtue of its negative charge, may confer acidic properties to the glycosylated compound. Such site(s) may be incorporated in the linker of the compounds of this invention and are preferably glycosylated by a cell during recombinant production of the polypeptide compounds (e.g., in mammalian cells such as CHO, BHK, COS). However, such sites may further be glycosylated by synthetic or semi-synthetic procedures known in the art.

### WSP Modification of a Compound

A process for preparing conjugation derivatives is also contemplated. Tumor cells, for example, exhibit epitopes not found on their normal counterparts. Such epitopes include, for example, different post-translational modifications resulting from their rapid proliferation. Thus, one aspect of this invention is a process comprising: a) selecting at least one randomized peptide that specifically binds to a target epitope; and b) preparing a pharmacologic agent comprising (i) at least one vehicle (Fc domain preferred), (ii) at least one amino acid sequence of the selected peptide or peptides, and (iii) an effector molecule.

In one aspect, the target epitope is a tumor-specific epitope or an epitope specific to a pathogenic organism. The effector molecule may be any of the above-noted conjugation partners and is preferably a radioisotope.

For obtaining a compound, with or without an Fc modification and/or linker(s), modified to include a covalently attached to WSP, any method described herein or otherwise known in the art is employed. By way of example and without limitation, a reductive alkylation chemical modification procedure method may be utilized. An alternative method for WSP modification is described in Francis et al., In: Stability of protein pharmaceuticals: in vivo pathways of degradation and strategies for protein stabilization (Eds. Ahern., T. and Manning, M. C.) Plenum, N.Y., 1991, is used. In still another aspect, the method described in Delgado et al., “Coupling of PEG to Protein By Activation With Tresyl Chloride, Applications In Immunoaffinity Cell Preparation”, In: Fisher et al., eds., Separations Using Aqueous Phase Systems, Applications In Cell Biology and Biotechnology, Plenum Press, N.Y. N.Y., 1989 pp. 211-213, which involves the use of tresyl chloride, which results in no linkage group between the WSP moiety and the TMP polypeptide moiety. This alternative method, however, may be difficult to use to produce therapeutic products as the use of tresyl chloride may produce toxic by-products. In other aspects, attachment of a WSP is effected through use of N-hydroxy succinimidyl esters of carboxymethyl methoxy polyethylene glycol, as well known in the art.

Depending on the method of WSP attachment chosen, the proportion of WSP molecules attached to the target peptide or protein molecule will vary, as will their concentrations in the reaction mixture. In general, the optimum ratio (in terms of efficiency of reaction in that there is no excess unreacted protein or polymer) is determined by the molecular weight of the WSP selected. In addition, when using methods that involve non-specific attachment and later purification of a desired species, the ratio may depend on the number of reactive groups (typically amino groups) available.

### Reductive Alkylation

In one aspect, covalent attachment of a WSP to a TMP, with or without Fc modification and with or without a linker, is carried out by reductive alkylation chemical modification procedures as provided herein to selectively modify the N-terminal α-amino group, and testing the resultant product for the desired biological characteristic, such as the biological activity assays provided herein.

Reductive alkylation for attachment of a WSP to a protein or peptide exploits differential reactivity of different types of primary amino groups (e.g., lysine versus the N-terminal) available for derivatization in a particular protein. Under the appropriate reaction conditions, substantially selective derivatization of the protein at the N-terminus with a carbonyl group containing polymer is achieved.

Using reductive alkylation, the reducing agent should be stable in aqueous solution and preferably be able to reduce only the Schiff base formed in the initial process of reductive alkylation. Reducing agents are selected from, and without limitation, sodium borohydride, sodium cyanoborohydride, dimethylamine borate, timethylamine borate and pyridine borate.

The reaction pH affects the ratio of polymer to protein to be used. In general, if the reaction pH is lower than the pKa of a target reactive group, a larger excess of polymer to protein will be desired. If the pH is higher than the target pKa, the polymer:protein ratio need not be as large (i.e., more reactive groups are available, so fewer polymer molecules are needed).

Accordingly, the reaction is performed in one aspect at a pH which allows one to take advantage of the pKa differences between the 8-amino groups of the lysine residues and that of the α-amino group of the N-terminal residue of the protein. By such selective derivatization, attachment of a water soluble polymer to a protein is controlled; the conjugation with the polymer takes place predominantly at the N-terminus of the protein and no significant modification of other reactive groups, such as the lysine side chain amino groups, occurs.

In one aspect, therefore, methods are provided for covalent attachment of a WSP to a target TMP and which provide a substantially homogenous preparation of WSP/protein conjugate molecules, in the absence of further extensive purification as is required using other chemical modification chemistries. More specifically, if polyethylene glycol is used, methods described allow for production of an N-terminally PEGylated protein lacking possibly antigenic linkage groups, i.e., the polyethylene glycol moiety is directly coupled to the protein moiety without potentially toxic by-products.

### Purification of a WSP-Modified Compound

The method of obtaining a substantially homogeneous WSP-TMP preparation is, in one aspect, by purification of a predominantly single species of modified TMP from a mixture of TMP species. By way of example, a substantially homogeneous TMP species is first separated by ion exchange chromatography to obtain material having a charge characteristic of a single species (even though other species having the same apparent charge may be present), and then the desired species is separated using size exclusion chromatography. Other methods are reported and contemplated by the invention, includes for example, PCT WO 90/04606, published May 3, 1990, which describes a process for fractionating a mixture of PEG-protein adducts comprising partitioning the PEG/protein adducts in a PEG-containing aqueous biphasic system.

Thus, one aspect of the present invention is a method for preparing a WSP-TMP conjugate comprised of (a) reacting a TMP having more than one amino group with a water soluble polymer moiety under reducing alkylation conditions, at a pH suitable to selectively activate the α-amino group at the amino terminus of the protein moiety so that said water soluble polymer selectively attaches to said α-amino group; and (b) obtaining the reaction product. Optionally, and particularly for a therapeutic product, the reaction products are separated from unreacted moieties.

### Bioassays

For assessing biological activity for a preparation of the invention, standard assays are contemplated, such as, for example and without limitation, those described in WO95/26746 entitled “Compositions and Methods for Stimulating Megakaryocyte Growth and Differentiation” and in U.S. Pat. No. 6,835,809, incorporated herein in its entirety.

In one such assay, normal mice of similar age are administered a preparation of the invention either with a bolus treatment or continuous delivery. Compounds administered include any preparation, whether in pharmaceutical composition for or not, with appropriate control(s).

Mice are bled at specified time points, generally with a minimum number of bleeds per week. At a set end time point, blood parameters, for example, white blood cells, red blood cells, hematocrit, hemoglobin, platelets, neutrophils are measured.

### Pharmaceutical Compositions

The present invention also provides methods of using pharmaceutical compositions of the inventive compounds. Such pharmaceutical compositions may be for administration for injection, or for oral, pulmonary, nasal, transdermal or other forms of administration. In general, the invention encompasses pharmaceutical compositions comprising effective amounts of a compound of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form. Implantable sustained release formulations are also contemplated, as are transdermal formulations.

### Oral Dosage Forms

Contemplated for use herein are oral solid dosage forms, which are described generally in Chapter 89 of Remington's Pharmaceutical Sciences (1990), 18th Ed., Mack Publishing Co. Easton Pa. 18042, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given in Chapter 10 of Marshall, K., Modern Pharmaceutics (1979), edited by G. S. Banker and C. T. Rhodes, herein incorporated by reference. In general, the formulation will include the inventive compound, and inert ingredients which allow for protection against the stomach environment, and release of the biologically active material in the intestine.

If necessary, the compounds may be chemically modified so that oral delivery is efficacious. Generally, the chemical modification contemplated is the attachment of at least one moiety to the compound molecule itself, where said moiety permits (a) inhibition of proteolysis; and (b) uptake into the blood stream from the stomach or intestine. Also desired is the increase in overall stability of the compound and increase in circulation time in the body. Moieties useful as covalently attached vehicles in this invention may also be used for this purpose. Examples of such moieties include: PEG, copolymers of ethylene glycol and propylene glycol, carboxymethyl cellulose, dextran, polyvinyl alcohol, polyvinyl pyrrolidone and polyproline. See, for example, Abuchowski and Davis, Soluble Polymer-Enzyme Adducts, Enzymes as Drugs (1981), Hocenberg and Roberts, eds., Wiley-Interscience, New York, N.Y., pp 367-83; Newmark, et al. (1982), J. Appl. Biochem. 4:185-9. Other polymers that could be used are poly-1,3-dioxolane and poly-1,3,6-tioxocane. In one aspect, PEG moieties are provided for pharmaceutical usage, as indicated above.

For oral delivery dosage forms, it is also possible to use a salt of a modified aliphatic amino acid, such as sodium N-(8-[2-hydroxybenzoyl]amino) caprylate (SNAC), as a carrier to enhance absorption of the therapeutic compounds of this invention. The clinical efficacy of a heparin formulation using SNAC has been demonstrated in a Phase II trial conducted by Emisphere Technologies. See U.S. Pat. No. 5,792,451, “Oral drug delivery composition and methods”.

The compounds of this invention can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the protein (or derivative) may be formulated (such as by liposome or microsphere encapsulation) and then further contained within an edible product, such as a refrigerated beverage containing colorants and flavoring agents.

One may dilute or increase the volume of the compound of the invention with an inert material. These diluents could include carbohydrates, especially mannitol, α-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An antifrictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the compound of this invention into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives may also be included in the formulation to enhance uptake of the compound. Additives potentially having this property are for instance the fatty acids oleic acid, linoleic acid and linolenic acid.

Controlled release formulation may be desirable. The compound of this invention could be incorporated into an inert matrix which permits release by either diffusion or leaching mechanisms e.g., gums. Slowly degenerating matrices may also be incorporated into the formulation, e.g., alginates, polysaccharides. Another form of a controlled release of the compounds of this invention is by a method based on the Oros therapeutic system (Alza Corp.), i.e., the drug is enclosed in a semipermeable membrane which allows water to enter and push drug out through a single small opening due to osmotic effects. Some enteric coatings also have a delayed release effect.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

### Pulmonary Delivery Forms

Also contemplated herein is pulmonary delivery of the present protein (or derivatives thereof). The protein (or derivative) is delivered to the lungs of a mammal while inhaling and traverses across the lung epithelial lining to the blood stream. (Other reports of this include Adjei et al., Pharma. Res. (1990) 7: 565-9; Adjei et al. (1990), Internatl. J. Pharmaceutics 63: 135-44 (leuprolide acetate); Braquet et al. (1989), J. Cardiovasc. Pharmacol. 13 (suppl.5): s.143-146 (endothelin-1); Hubbard et al. (1989), Annals Int. Med. 3: 206-12 (α1-antitrypsin); Smith et al. (1989), J. Clin. Invest. 84: 1145-6 (α1-proteinase); Oswein et al. (March 1990), “Aerosolization of Proteins”, Proc. Symp. Resp. Drug Delivery II, Keystone, Colo. (recombinant human growth hormone); Debs et al. (1988), J. Immunol. 140: 3482-8 (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the inventive compound. Typically, each formulation is specific to the type of device employed and may involve the use of an appropriate propellant material, in addition to diluents, adjuvants and/or carriers useful in therapy.

The inventive compound should most advantageously be prepared in particulate form with an average particle size of less than 10 μm (or microns), most preferably 0.5 to 5 μm, for most effective delivery to the distal lung.

Pharmaceutically acceptable carriers include carbohydrates such as trehalose, mannitol, xylitol, sucrose, lactose, and sorbitol. Other ingredients for use in formulations may include DPPC, DOPE, DSPC and DOPC. Natural or synthetic surfactants may be used. PEG may be used (even apart from its use in derivatizing the protein or analog). Dextrans, such as cyclodextran, may be used. Bile salts and other related enhancers may be used. Cellulose and cellulose derivatives may be used. Amino acids may be used, such as use in a buffer formulation.

Also, the use of liposomes, microcapsules or microspheres, inclusion complexes, or other types of carriers is contemplated.

Formulations suitable for use with a nebulizer, either jet or ultrasonic, will typically comprise the inventive compound dissolved in water at a concentration of about 0.1 to 25 mg of biologically active protein per mL of solution. The formulation may also include a buffer and a simple sugar (e.g., for protein stabilization and regulation of osmotic pressure). The nebulizer formulation may also contain a surfactant, to reduce or prevent surface induced aggregation of the protein caused by atomization of the solution in forming the aerosol.

Formulations for use with a metered-dose inhaler device will generally comprise a finely divided powder containing the inventive compound suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the inventive compound and may also include a bulking agent, such as lactose, sorbitol, sucrose, mannitol, trehalose, or xylitol in amounts which facilitate dispersal of the powder from the device, e.g., 50 to 90% by weight of the formulation.

### Nasal Delivery Forms

Nasal delivery of the inventive compound is also contemplated. Nasal delivery allows the passage of the protein to the blood stream directly after administering the therapeutic product to the nose, without the necessity for deposition of the product in the lung. Formulations for nasal delivery include those with dextran or cyclodextran. Delivery via transport across other mucous membranes is also contemplated.

### Buccal Delivery Forms

Buccal delivery of the inventive compound is also contemplated. Buccal delivery formulations are known in the art for use with peptides.

### Dosages

The dosage regimen involved in a method for treating the above-described conditions will be determined by the attending physician, considering various factors which modify the action of drugs, e.g. the age, condition, body weight, sex and diet of the patient, the severity of any infection, time of administration and other clinical factors. Generally, the daily regimen should be in the range of 0.1-1000 micrograms of the inventive compound per kilogram of body weight, preferably 0.1-150 micrograms per kilogram.

Provided herein are pharmaceutical compositions comprising preparations of the invention. Such pharmaceutical compositions may be for administration for injection, or for oral, nasal, transdermal or other forms of administration, including, e.g., by intravenous, intradermal, intramuscular, intramammary, intraperitoneal, intrathecal, intraocular, retrobulbar, intrapulmonary (e.g., aerosolized drugs) or subcutaneous injection (including depot administration for long term release); by sublingual, anal, vaginal, or by surgical implantation, e.g., embedded under the splenic capsule, brain, or in the cornea. The treatment may consist of a single dose or a plurality of doses over a period of time. In general, comprehended by the invention are pharmaceutical compositions comprising effective amounts of a compound of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. The pharmaceutical compositions optionally may include still other pharmaceutically acceptable liquid, semisolid, or solid diluents that serve as pharmaceutical vehicles, excipients, or media, including but are not limited to, polyoxyethylene sorbitan monolaurate, magnesium stearate, methyl- and propylhydroxybenzoate, starches, sucrose, dextrose, gum acacia, calcium phosphate, mineral oil, cocoa butter, and oil of theobroma. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form. Implantable sustained release formulations are also contemplated, as are transdermal formulations.

The therapeutic methods, compositions and compounds of the present invention may also be employed, alone or in combination with other cytokines, soluble c-Mpl receptor, hematopoietic factors, interleukins, growth factors or antibodies in the treatment of disease states characterized by other symptoms as well as platelet deficiencies. It is anticipated that the preparations of the invention will prove useful in treating some forms of thrombocytopenia in combination with general stimulators of hematopoiesis, such as IL-3 or GM-CSF. Other megakaryocytic stimulatory factors, i.e., meg-CSF, stem cell factor (SCF), leukemia inhibitory factor (LIF), oncostatin M (OSM), or other molecules with megakaryocyte stimulating activity may also be employed with Mpl ligand.

Additional exemplary cytokines or hematopoietic factors for such co-administration include IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor receptor, TNF, including TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

It may further be useful to administer, either simultaneously or sequentially, an effective amount of a soluble mammalian c-Mpl, which appears to have an effect of causing megakaryocytes to fragment into platelets once the megakaryocytes have reached mature form. Thus, administration of a preparation of the invention (to enhance the number of mature megakaryocytes) followed by administration of the soluble c-Mpl (to inactivate the ligand and allow the mature megakaryocytes to produce platelets) is expected to be a particularly effective means of stimulating platelet production. The dosage recited above would be adjusted to compensate for such additional components in the therapeutic composition. Progress of the treated patient can be monitored by conventional methods.

### Therapeutic Uses

For the compounds herein, one can utilize such standard assays as those described in WO95/26746 entitled “Compositions and Methods for Stimulating Megakaryocyte Growth and Differentiation”. In vivo assays also appear in the Examples hereinafter.

The conditions to be treated are generally those that involve an existing megakaryocyte/platelet deficiency or an expected megakaryocyte/platelet deficiency (e.g., because of planned surgery or platelet donation). Such conditions will usually be the result of a deficiency (temporary or permanent) of active thrombopoietin in vivo. The generic term for platelet deficiency is thrombocytopenia, and the methods and compositions of the present invention are generally available for treating thrombocytopenia in patients in need thereof.

Thrombocytopenia (platelet deficiencies) may be present for various reasons, including chemotherapy and other therapy with a variety of drugs, radiation therapy, surgery, accidental blood loss, and other specific disease conditions. Exemplary specific disease conditions that involve thrombocytopenia and may be treated in accordance with this invention are: aplastic anemia; idiopathic or immune thrombocytopenia (ITP), including idiopathic thrombocytopenic purpura associated with breast cancer; HIV associated ITP and HIV-related thrombotic thrombocytopenic purpura; metastatic tumors which result in thrombocytopenia; systemic lupus erythematosus; including neonatal lupus syndrome splenomegaly; Fanconi's syndrome; vitamin B12 deficiency; folic acid deficiency; May-Hegglin anomaly; Wiskott-Aldrich syndrome; chronic liver disease; myelodysplastic syndrome associated with thrombocytopenia; paroxysmal nocturnal hemoglobinuria; acute profound thrombocytopenia following C7E3 Fab (Abciximab) therapy; alloimmune thrombocytopenia, including maternal alloimmune thrombocytopenia; thrombocytopenia associated with antiphospholipid antibodies and thrombosis; autoimmune thrombocytopenia; drug-induced immune thrombocytopenia, including carboplatin-induced thrombocytopenia, heparin-induced thrombocytopenia; fetal thrombocytopenia; gestational thrombocytopenia; Hughes' syndrome; lupoid thrombocytopenia; accidental and/or massive blood loss; myeloproliferative disorders; thrombocytopenia in patients with malignancies; thrombotic thrombocytopenia purpura, including thrombotic microangiopathy manifesting as thrombotic thrombocytopenic purpura/hemolytic uremic syndrome in cancer patients; autoimmune hemolytic anemia; occult jejunal diverticulum perforation; pure red cell aplasia; autoimmune thrombocytopenia; nephropathia epidemica; rifampicin-associated acute renal failure; Paris-Trousseau thrombocytopenia; neonatal alloimmune thrombocytopenia; paroxysmal nocturnal hemoglobinuria; hematologic changes in stomach cancer; hemolytic uremic syndromes in childhood; hematologic manifestations related to viral infection including hepatitis A virus and CMV-associated thrombocytopenia. Other hepatic diseases or conditions that involve thrombocytopenia and may be treated in accordance with this invention, in addition to viral hepatitis A (HAV) include, but are not limited to, alcoholic hepatitis, autoimmune hepatitis, drug-induced hepatitis, epidemic hepatitis, infectious hepatitis, long-incubation hepatitis, noninfectious hepatitis, serum hepatitis, short-incubation hepatitis, toxic hepatitis, transfusion hepatitis, viral hepatitis B (HBV), viral hepatitis C(HCV), viral hepatitis D (HDV), delta hepatitis, viral hepatitis E (HEV), viral hepatitis F (HFV), viral hepatitis G (HGV), liver disease, inflammation of the liver, hepatic failure, and other hepatic disease. Also, certain treatments for AIDS result in thrombocytopenia (e.g., AZT). Certain wound healing disorders might also benefit from an increase in platelet numbers.

With regard to anticipated platelet deficiencies, e.g., due to future surgery, a compound of the present invention could be administered several days to several hours prior to the need for platelets. With regard to acute situations, e.g., accidental and massive blood loss, a compound of this invention could be administered along with blood or purified platelets.

The compounds of this invention may also be useful in stimulating certain cell types other than megakaryocytes if such cells are found to express Mpl receptor. Conditions associated with such cells that express the Mpl receptor, which are responsive to stimulation by the Mpl ligand, are also within the scope of this invention.

In addition, the compounds of this invention may be used in any situation in which production of platelets or platelet precursor cells is desired, or in which stimulation of the c-Mpl receptor is desired. Thus, for example, the compounds of this invention may be used to treat any condition in a mammal wherein there is a need of platelets, megakaryocytes, and the like. Such conditions are described in detail in the following exemplary sources: WO95/26746; WO95/21919; WO95/18858; WO95/21920 and are incorporated herein.

The compounds of this invention may also be useful in maintaining the viability or storage life of platelets and/or megakaryocytes and related cells. Accordingly, it could be useful to include an effective amount of one or more such compounds in a composition containing such cells.

The therapeutic methods, compositions and compounds of the present invention may also be employed, alone or in combination with other cytokines, soluble Mpl receptor, hematopoietic factors, interleukins, growth factors or antibodies in the treatment of disease states characterized by other symptoms as well as platelet deficiencies. It is anticipated that the inventive compound will prove useful in treating some forms of thrombocytopenia in combination with general stimulators of hematopoiesis, such as IL-3 or GM-CSF. Other megakaryocytic stimulatory factors, i.e., meg-CSF, stem cell factor (SCF), leukemia inhibitory factor (LIF), oncostatin M (OSM), or other molecules with megakaryocyte stimulating activity may also be employed with Mpl ligand. Additional exemplary cytokines or hematopoietic factors for such co-administration include IL-1 alpha, IL-1 beta, IL-2, IL-3, IL-4, IL-5, IL-6, IL-11, colony stimulating factor-1 (CSF-1), SCF, GM-CSF, granulocyte colony stimulating factor (G-CSF), EPO, interferon-alpha (IFN-alpha), consensus interferon, IFN-beta, or IFN-gamma. It may further be useful to administer, either simultaneously or sequentially, an effective amount of a soluble mammalian Mpl receptor, which appears to have an effect of causing megakaryocytes to fragment into platelets once the megakaryocytes have reached mature form. Thus, administration of an inventive compound (to enhance the number of mature megakaryocytes) followed by administration of the soluble Mpl receptor (to inactivate the ligand and allow the mature megakaryocytes to produce platelets) is expected to be a particularly effective means of stimulating platelet production. The dosage recited above would be adjusted to compensate for such additional components in the therapeutic composition. Progress of the treated patient can be monitored by conventional methods.

In cases where the inventive compounds are added to compositions of platelets and/or megakaryocytes and related cells, the amount to be included will generally be ascertained experimentally by techniques and assays known in the art. An exemplary range of amounts is 0.1 μg-1 mg inventive compound per 10⁶ cells.

In addition to therapeutic uses, the compounds of the present invention are useful in diagnosing diseases characterized by dysfunction of their associated protein of interest. In one embodiment, a method of detecting in a biological sample a protein of interest (e.g., a receptor) that is capable of being activated comprising the steps of: (a) contacting the sample with a compound of this invention; and (b) detecting activation of the protein of interest by the compound. The biological samples include tissue specimens, intact cells, or extracts thereof. The compounds of this invention may be used as part of a diagnostic kit to detect the presence of their associated proteins of interest in a biological sample. Such kits employ the compounds of the invention having an attached label to allow for detection. The compounds are useful for identifying normal or abnormal proteins of interest.

It is understood that the application of the teachings of the present invention to a specific problem or situation will be within the capabilities of one having ordinary skill in the art in light of the teachings contained herein. Examples of the products of the present invention and representative processes for their isolation, use, and manufacture appear below.

### Examples

I. The following sets forth exemplary methods for making some of the compounds of the first group disclosed herein.

### A. Materials and Methods

All amino acid derivatives (all of L-configurations) and resins used in peptide synthesis may be purchased from Novabiochem. Peptide synthesis reagents (DCC, HOBt, etc.) may be purchased in the solution forms from Applied Biosystems, Inc. The two PEG derivatives are from Shearwater Polymers, Inc. All solvents (dichloromethane, N-methylpyrrolidinone, methanol, acetonitrile) are from EM Sciences. Analytical HPLC is run on a Beckman system with a Vydac column (0.46 cm×25 cm, C18 reversed phase, 5 mm), at a flow rate of 1 ml/min and with dual UV detection at 220 and 280 nm. Linear gradients are used for all HPLC operations with two mobile phases: Buffer A—H₂O (0.1% TFA) and Buffer B—acetonitrile (0.1% TFA). The TPO mimetics referred to herein are provided in Tables 1-3,5,7,8, and 11

### Peptide Synthesis

Peptides are prepared using a variety of methods known in the art, including the well established stepwise solid phase synthesis method. Solid-phase synthesis with Fmoc chemistry is carried out using an ABI Peptide Synthesizer. Typically, peptide synthesis begins with a preloaded Wang resin on a 0.1 mmol scale. Fmoc deprotection is carried out with the standard piperidine protocol. The coupling is effected using DCC/HOBt. Side-chain protecting groups were: Glu(O-t-Bu), Thr(t-Bu), Arg(Pbf), Gln(Trt), Trp(t-Boc) and Cys(Trt). For the first peptide precursor for pegylation, Dde is used for side chain protection of the Lys on the linker and Boc-11e-OH is used for the last coupling. Dde is removed by using anhydrous hydrazine (2% in NMP, 3×2 min), followed by coupling with bromoacetic anhydride preformed by the action of DCC. For peptide 18, the cysteine side chain in the linker is protected by a trityl group. The final deprotection and cleavage of all peptidyl-resins is effected at RT for 4 hr, using trifluoroacetic acid (TFA) containing 2.5% H₂O, 5% phenol, 2.5% triisopropylsilane and 2.5% thioanisole. After removal of TFA, the cleaved peptide is precipitated with cold anhydrous ether. Disulfide formation of the cyclic peptide is performed directly on the crude material by using 15% DMSO in H₂O (pH 7.5). All crude peptides are purified by preparative reverse phase HPLC and the structures are confirmed by ESI-MS and amino acid analysis.

Peptides are also prepared by phage library generation. The details on library generation methods and phage panning methods were described previously (see PCT/US02/32657 and US/2003/0176352). Phage panning methods are also performed using biotinylated MPL in the range of 10-0.01 μg per 100 μL of Streptavidin Dynabeads (Dynal, Lake Success, N.Y.). After phage are bound to the beads, they are washed 20-50 times before they are eluted. Phage ELISA for TPO-like activity and sequencing analysis are performed as described previously (PCT/US02/32657 and US/2003/0176352).

Alternatively, all peptides described in the application could also be prepared by using the t-Boc chemistry. In this case, the starting resins would be the classic Merrifield or Pam resin, and side chain protecting groups would be: Glu(OBz1), Thr(Bz1), Arg(Tos), Trp(CHO), Cys(p-MeBz1). Hydrogen fluoride (HF) would be used for the final cleavage of the peptidyl resins.

All peptides and tandem dimeric peptides described in herein that have linkers composed of natural amino acids can also be prepared by recombinant DNA technology.

### Pegylation

A novel, convergent strategy for the pegylation of synthetic peptides was developed which consists of combining, through forming a conjugate linkage in solution, a peptide and a PEG moiety, each bearing a special functionality that is mutually reactive toward the other. The precursor peptides can be easily prepared with the conventional solid phase synthesis as described above. As described below, these peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The pegylated peptides can be easily purified by preparative HPLC and characterized by analytical HPLC, amino acid analysis and laser desorption mass spectrometry.

### Bioactivity Assay

The TPO in vitro bioassay is a mitogenic assay utilizing an IL-3 dependent clone of murine 32D cells that have been transfected with human mpl receptor. This assay is described in greater detail in WO 95/26746. Cells are maintained in MEM medium containing 10% Fetal Clone II and 1 ng/ml mIL-3. Prior to sample addition, cells are prepared by rinsing twice with growth medium lacking mIL-3. An extended twelve point TPO standard curve is prepared, ranging from 3333 to 39 pg/ml. Four dilutions, estimated to fall within the linear portion of the standard curve, (1000 to 125 pg/ml), are prepared for each sample and run in triplicate. A volume of 100 μl of each dilution of sample or standard is added to appropriate wells of a 96 well microtiter plate containing 10,000 cells/well. After forty-four hours at 37° C. and 10% CO₂, MTS (a tetrazolium compound which is bioreduced by cells to a formazan) is added to each well. Approximately six hours later, the optical density is read on a plate reader at 490 nm. A dose response curve (log TPO concentration vs. O.D.-Background) is generated and linear regression analysis of points which fall in the linear portion of the standard curve is performed. Concentrations of unknown test samples are determined using the resulting linear equation and a correction for the dilution factor. The TPO in vivo bioassay tests for platelet production in mice after administration of the compounds of the invention.

### Abbreviations

HPLC: high performance liquid chromatography; ESI-MS: Electron spray ionization mass spectrometry; MALDI-MS: Matrix-assisted laser desorption ionization mass spectrometry; PEG: Poly(ethylene glycol). All amino acids are represented in the standard three-letter or single-letter codes. t-Boc: tert-Butoxycarbonyl; tBu: tert-Butyl; Bzl: Benzyl; DCC: Dicylcohexylcarbodiimide; HOBt: 1-Hydroxybenzotriazole; NMP: N-methyl-2-pyrrolidinone; Pbf: 2,2,4,6,7-pendamethyldihydro-benzofuran-5-sulfonyl; Trt: trityl; Dde: 1-(4,4-dimethyl-2,6-dioxo-cyclohexylidene)ethyl.

### B. Results

### TMP Monomers, Multimers and FC-TMP Fusion Proteins

A series of TPO-mimetic peptides and TPO-mimetic fusion proteins were synthesized. TPO-mimetic peptides are readily synthesized by conventional solid phase peptide synthesis methods (Merrifiled, R. B., Journal of the American Chemical Society 85:2149 (1963)) with either Fmoc or t-Boc chemistry, by phage peptide library synthesis, or any other method known in the art. In such libraries, random peptide sequences are displayed by fusion with coat proteins of filamentous phage. Typically, the displayed peptides are affinity-eluted against an antibody-immobilized extracellular domain of a receptor. The retained phages may be enriched by successive rounds of affinity purification and repropagation. The best binding peptides may be sequenced to identify key residues within one or more structurally related families of peptides.

TABLE 1
TPO-MIMETIC PEPTIDES

| | |
|---|---|
| AMINO ACID SEQUENCE | SEQ ID NO: |

| | |
|---|---|
| QGCSSGGPTQREWLQCRRMQHS | 34 |
| QGCSSGGPTLREWQQCRRMQHS | 35 |
| QGCSWGGPTLKIWLQCVRAKHS | 36 |
| QGCSWGGPTLKNWLQCVRAKHS | 37 |
| QGCSWGGPTLKLWLQCVRAKHS | 38 |
| QGCSWGGPTLKHWLQCVRAKHS | 39 |
| QGGCRSGPTNREWLACREVQHS | 40 |
| QGTCEQGPTLRQWPLCRQGRHS | 41 |
| QGTCEQGPTLRLWLLCRQGRHS | 42 |
| QGTCEQGPTLRIWLLCRQGRHS | 43 |

Table 2 summarizes relative activities (% control activity) of some of the TPO-mimetic fusion proteins of the invention in terms of relative potencies based on in vitro assays as described above. An Fc molecule is fused at either the N-terminus or the C-terminus of the peptide. Some TPO-mimetics comprise an Fc molecule connected at the N-terminus of a dimer of the peptide (see, e.g., Fc-2-(SEQ ID NO: 35)). “Fc-2-peptide” and “Fc-2×-peptide” are used interchangeably to indicate that an Fc molecule is fused at the N-terminus of two copies of a peptide connected in tandem. As with all of the TPO-mimetic compounds, the peptide may be attached at the C-terminus of the Fc molecule with a linker/spacer or inserted into an Fc-Loop, optionally with the use of symmetric or asymmetric linkers/spacers.

TABLE 2
TPO-MIMETIC FUSION PROTEINS

| TPO-Mimetic | % CONTROL ACTIVITY | % ERROR |
|---|---|---|
| Fc-(SEQ ID NO: 35) | 88.5 | 24.9 |
| Fc-2-(SEQ ID NO: 35) | 80.5 | 14.9 |
| Fc-(SEQ ID NO: 37) | 78.6 | 19.1 |
| Fc-2-(SEQ ID NO: 34) | 74.8 | 11.4 |
| Fc-(SEQ ID NO: 38) | 67.4 | 16.0 |
| Fc-(SEQ ID NO: 39) | 60.9 | 7.7 |
| (SEQ ID NO: 36)-Fc | 45.7 | 11.2 |
| Fc-(SEQ ID NO: 36) | 40.5 | 9.8 |
| Fc-2-(SEQ ID NO: 38) | 37.7 | 8.7 |
| Fc-(SEQ ID NO: 41) | 26.2 | 6.1 |
| Fc-(SEQ ID NO: 34) | 25.8 | 6.1 |
| (SEQ ID NO: 41)-Fc | 24.6 | 6.1 |
| Fc-(SEQ ID NO: 39) | 23.2 | 2.5 |
| Fc-2-(SEQ ID NO: 40) | 22.0 | 8.5 |

Table 3 sets out still other TPO-mimetic peptides having c-mpl receptor binding activity. These peptides are contemplated for use alone or as TPO-mimetic fusion proteins, wherein the TPO-mimetic peptide is fused to either an N-terminus of an Fc region or within an Fc-Loop, a modified Fc molecule. Fc-Loops are described herein and in U.S. Patent Application Publication No. US2006/0140934 incorporated herein by reference in its entirety.

TABLE 3
TPO-MIMETIC PEPTIDES

| AMINO ACID SEQUENCE | SEQ ID NO: |
|---|---|
| QGCSSGGPTLREWQQCRRMQHS | 35 |
| QGCSSGGPTLREWQQCVRMQHS | 5 |
| QGCSSGGPTLREWQQCRRAQHS | 6 |
| QGCSSGGPTLREWQQCVRAQHS | 7 |
| QGCSSGGPTLREWQQCVQAQHS (FcL2) | 11 |
| QGCSSGGPTLREWQQCVGAQHS (FcL3) | 12 |
| QGCSSGGPTLREWQQCVHAQHS (FcL4) | 13 |
| QGCSSGGPTLREWQQCQGAQHS (FcL5) | 14 |
| QGCSSGGPTLREWQQCVRPQHS (FcL6) | 15 |
| QGCSSGGPTLREWQQCFRPQHS (FcL7) | 16 |
| QGCSSGGPTLREWQQCFKAQHS (FcL8) | 17 |
| QGCSSGGPTLREWQQCVKPQHS (FcL9) | 18 |
| QGCSSGGPTLREWQQCVRAQHS (FcL10) | 19 |
| QGCSSGGPTLREWQQCRPAQHS (FcL11) | 20 |
| QGCSSGGPTLREWQQCRRPQHS (FcL12) | 21 |
| QGCSSGGPTLREWQQCQRAQHS (FcL13) | 22 |
| QGCSSGGPTLREWQQCSRAQHS (FcL14) | 23 |

### FC-Loops

As set out above, all of the peptides discussed herein are contemplated for use alone or as TPO-mimetic fusion proteins, wherein the TPO-mimetic peptide is fused to either an N-terminus of an Fc region or within an Fc-Loop, a modified Fc molecule.

Fc-Loops comprising a TPO-mimetic peptide are prepared in a process in which at least one biologically active peptide is incorporated as an internal sequence into an Fc domain. Such an internal sequence may be added by insertion (i.e., between amino acids in the previously existing Fc domain) or by replacement of amino acids in the previously existing Fc domain (i.e., removing amino acids in the previously existing Fc domain and adding peptide amino acids). In the latter case, the number of peptide amino acids added need not correspond to the number of amino acids removed from the previously existing Fc domain. For example, in one aspect, a molecule in which 10 amino acids are removed and 15 amino acids are added is provided. Pharmacologically active compounds provided are prepared by a process comprising: a) selecting at least one peptide that modulates the activity of a protein of interest; and b) preparing a pharmacologic agent comprising an amino acid sequence of the selected peptide as an internal sequence of an Fc domain. This process may be employed to modify an Fc domain that is already linked through an N- or C-terminus or sidechain to a peptide, e.g., as described in U.S. Pat. App. Nos. 2003/0195156, 2003/0176352, 2003/0229023, and 2003/0236193, and international publication numbers WO 00/24770 and WO 04/026329. The process described in U.S. Patent Application Publication No. US2006/0140934 may also be employed to modify an Fc domain that is part of an antibody. In this way, different molecules can be produced that have additional functionalities, such as a binding domain to a different epitope or an additional binding domain to the precursor molecule's existing epitope. Molecules comprising an internal peptide sequence are also referred to as “Fc internal peptibodies” or “Fc internal peptide molecules.”

The Fc internal peptide molecules may include more than one peptide sequence in tandem in a particular internal region, and they may include further peptides in other internal regions. While the putative loop regions are preferred, insertions in any other non-terminal domains of the Fc are also considered part of this invention. Variants and derivatives of the above compounds (described below) are also encompassed by this invention.

The compounds of this invention may be prepared by standard synthetic methods, recombinant DNA techniques, or any other methods of preparing peptides and fusion proteins.

A use contemplated for Fc internal peptide molecules is as a therapeutic or a prophylactic agent. A selected peptide may have activity comparable to—or even greater than—the natural ligand mimicked by the peptide. In addition, certain natural ligand-based therapeutic agents might induce antibodies against the patient's own endogenous ligand. In contrast, the unique sequence of the vehicle-linked peptide avoids this pitfall by having little or typically no sequence identity with the natural ligand. Furthermore, the Fc internal peptibodies may have advantages in refolding and purification over N- or C-terminally linked Fc molecules. Further still, Fc internal peptibodies may be more stable in both thermodynamically, due to the stabilization of chimeric domains, and chemically, due to increased resistance to proteolytic degradation from amino- and carboxy-peptidases. Fc internal peptibodies may also exhibit improved pharmacokinetic properties.

In one embodiment, the invention includes Fc-Loop-QGCSSGGPTLREWQQCRRMQHS (SEQ ID NO: 35) wherein the peptide sequence of SEQ ID NO: 35 is inserted in the Fc molecule (SEQ ID NO: 24) in the loop region between amino acids 139 (Leu) and 140 (Thr) using a linker. In one aspect, the linker comprises four glycine residues at the N-terminus of the amino acid sequence of SEQ ID NO: 35. In another aspect, the linker comprises two glycine residues at the N-terminus and two glycine residues at the C-terminus of SEQ ID NO: 35.

In another embodiment, the invention includes Fc-Loop-QGCSSGGPTLREWQQCVRMQHS (SEQ ID NO: 5) wherein the peptide sequence of SEQ ID NO: 35 is inserted in the Fc molecule (SEQ ID NO: 24) in the loop region between amino acids 139 (L) and 140 (Thr) using a linker. In one aspect, the linker comprises four glycine residues at the N-terminus of the amino acid sequence of SEQ ID NO: 5. In another aspect, the linker comprises two glycine residues at the N-terminus and two glycine residues at the C-terminus of SEQ ID NO: 5.

Other linkers, as discussed in U.S. Patent Application Publication No. US2006/0140934, are also contemplated for use in modifying Fc-Loop molecules in this embodiment.

FC-Loop Insertion Sites

As set out above, all of the peptides discussed herein are contemplated for use alone or as TPO-mimetic fusion proteins, wherein the TPO-mimetic peptide is fused to either an N-terminus of an Fc region or within an Fc-Loop, a modified Fc molecule. Fc-Loops are described in U.S. Patent Application Publication No. US2006/0140934 incorporated herein by reference in its entirety. Preferred internal sites for peptide addition into an Fc-Loop are shown in boldface below:

(SEQ ID NO: 24)

| | |
|---|---|
| 1 | MDKTHTCPPC PAPELLGGPS VFLFPPKPKD TLMISRTPEV |
| | TCVVVDVSHE |
| 51 | DPEVKFNWYV DGVEVHNAKT KPREEQYNST YRVVSVLTVL |
| | HQDWLNGKEY |
| 101 | KCKVSNKALP APIEKTISKA KGQPREPQVY TLPPSRDELT |
| | KNQVSLTCLV |
| 151 | KGFYPSDIAV EWESNGQPEN NYKTTPPVLD SDGSFFLYSK |
| | LTVDKSRWQQ |
| 201 | GNVFSCSVMH EALHNHYTQK SLSLSPGK. |

Particularly preferred sites are the insertion sites (H49/E50), (Y77/N78), (K107/A108), (L139/T140), (E169/N170), (S181/D182), and (G201/N202) of SEQ ID NO: 24. Most preferable are the insertion site (L139/T140) of SEQ ID NO: 24 and two additional loops in the CH2 domain (H49/E50) and (Y77/N78).

In one embodiment, a TPO-mimetic peptide is inserted into the human IgG1 Fc-Loop domain between Leu139 and Thr140 of SEQ ID NO: 24 and includes 2 Gly residues as linkers flanking either side of the inserted peptide.

Other exemplary amino acid sequences of human Fc regions from IgA, IgM and IgG subtypes (SEQ ID NOS: 25 to 32), as set out in Table 4 below, may also be used in the invention in addition to the Fc region set out in SEQ ID NO: 24. A consensus sequence is set out in (SEQ ID NO: 33).

TABLE 4
AMINO ACID SEQUENCES OF ADDITIONAL HUMAN FC REGIONS

| | SEQ |
|---|---|
| | ID |
| AMINO ACID SEQUENCE | NO: |
| Ala Gly Lys Ser Val Thr Cys His Val Lys His Tyr Thr Asn Pro Ser Gln Asp Val Thr Val Pro Cys Pro | 25 |
| Val Pro Ser Thr Pro Pro Thr Pro Ser Pro Ser Thr Pro Pro Thr Pro Ser Pro Ser Cys Cys His Pro Arg | |
| Leu Ser Leu His Arg Pro Ala Leu Glu Asp Leu Leu Leu Gly Ser Glu Ala Asn Leu Thr Cys Thr Leu Thr | |
| Gly Leu Arg Asp Ala Ser Gly Val Thr Phe Thr Trp Thr Pro Ser Ser Gly Lys Ser Ala Val Gln Gly Pro | |
| Pro Glu Arg Asp Leu Cys Gly Cys Tyr Ser Val Ser Ser Val Leu Pro Gly Cys Ala Glu Pro Trp Asn His | |
| Gly Lys Thr Phe Thr Cys Thr Ala Ala Tyr Pro Glu Ser Lys Thr Pro Leu Thr Ala Thr Leu Ser Lys Ser | |
| Gly Asn Thr Phe Arg Pro Glu Val His Leu Leu Pro Pro Pro Ser Glu Glu Leu Ala Leu Asn Glu Leu Val | |
| Thr Leu Thr Cys Leu Ala Arg Gly Phe Ser Pro Lys Asp Val Leu Val Arg Trp Leu Gln Gly Ser Gln Glu | |
| Leu Pro Arg Glu Lys Tyr Leu Thr Trp Ala Ser Arg Gln Glu Pro Ser Gln Gly Thr Thr Thr Phe Ala Val | |
| Thr Ser Ile Leu Arg Val Ala Ala Glu Asp Trp Lys Lys Gly Asp Thr Phe Ser Cys Met Val Gly His Glu | |
| Ala Leu Pro Leu Ala Phe Thr Gln Lys Thr Ile Asp Arg Leu Ala Gly Lys Pro Thr His Val Asn Val Ser | |
| Val Val Met Ala Glu Val Asp Gly Thr Cys Tyr | |
| Asp Gly Lys Ser Val Thr Cys His Val Lys His Tyr Thr Asn Pro Ser Gln Asp Val Thr Val Pro Cys Pro | 26 |
| Val Pro Pro Pro Pro Pro Cys Cys His Pro Arg Leu Ser Leu His Arg Pro Ala Leu Glu Asp Leu Leu Leu | |
| Gly Ser Glu Ala Asn Leu Thr Cys Thr Leu Thr Gly Leu Arg Asp Ala Ser Gly Ala Thr Phe Thr Trp Thr | |
| Pro Ser Ser Gly Lys Ser Ala Val Gln Gly Pro Pro Glu Arg Asp Leu Cys Gly Cys Tyr Ser Val Ser Ser | |
| Val Leu Pro Gly Cys Ala Gln Pro Trp Asn His Gly Glu Thr Phe Thr Cys Thr Ala Ala His Pro Glu Leu | |
| Lys Thr Pro Leu Thr Ala Asn Ile Thr Lys Ser Gly Asn Thr Phe Arg Pro Glu Val His Leu Leu Pro Pro | |
| Pro Ser Glu Glu Leu Ala Leu Asn Glu Leu Val Thr Leu Thr Cys Leu Ala Arg Gly Phe Ser Pro Lys Asp | |
| Val Leu Val Arg Trp Leu Gln Gly Ser Gln Glu Leu Pro Arg Glu Lys Tyr Leu Thr Trp Ala Ser Arg Gln | |
| Glu Pro Ser Gln Gly Thr Thr Thr Phe Ala Val Thr Ser Ile Leu Arg Val Ala Ala Glu Asp Trp Lys Lys | |
| Gly Asp Thr Phe Ser Cys Met Val Gly His Glu Ala Leu Pro Leu Ala Phe Thr Gln Lys Thr Ile Asp Arg | |
| Leu Ala Gly Lys Pro Thr His Val Asn Val Ser Val Val Met Ala Glu Val Asp Gly Thr Cys Tyr | |
| Glu Gly Lys Gln Val Gly Ser Gly Val Thr Thr Asp Gln Val Gln Ala Glu Ala Lys Glu Ser Gly Pro Thr | 27 |
| Thr Tyr Lys Val Thr Ser Thr Leu Thr Ile Lys Glu Asp His Arg Gly Leu Thr Phe Gln Gln Asn Ala Ser | |
| Ser Met Cys Val Pro Asp Gln Asp Thr Ala Ile Arg Val Phe Ala Ile Pro Pro Ser Phe Ala Ser Ile Phe | |
| Leu Thr Lys Ser Thr Lys Leu Thr Cys Leu Val Thr Asp Leu Thr Thr Tyr Asp Ser Val Thr Ile Ser Trp | |
| Asn Ser Gly Glu Arg Phe Thr Cys Thr Val Thr His Thr Asp Leu Pro Ser Pro Leu Lys Gln Thr Ile Ser | |
| Arg Pro Lys Gly Val Ala Leu His Arg Pro Asp Val Tyr Leu Leu Pro Pro Ala Arg Glu Gln Leu Asn Leu | |
| Arg Glu Ser Ala Thr Ile Thr Cys Leu Val Thr Gly Phe Ser Pro Ala Asp Val Phe Val Gln Trp Met Gln | |
| Arg Gly Gln Pro Leu Ser Pro Glu Lys Tyr Val Thr Ser Ala Pro Met Pro Glu Pro Gln Ala Pro Gly Arg | |
| Tyr Phe Ala His Ser Ile Leu Thr Val Ser Glu Glu Glu Trp Asn Thr Gly Glu Thr Tyr Thr Cys Val Ala | |
| His Asp Ala Leu Pro Asn Arg Val Thr Glu Arg Thr Val Asp Lys Ser Thr Gly Lys Pro Thr Leu Tyr Asn | |
| Val Ser Leu Val Met Ser Asp Thr Ala Gly Thr Cys Tyr | |
| Glu Pro Lys Ser Cys Asp Lys Thr His Thr Cys Pro Pro Cys Pro Ala Pro Glu Leu Leu Gly Gly Pro Ser | 28 |
| Val Phe Leu Phe Pro Pro Lys Pro Lys Asp Thr Leu Met Ile Ser Arg Thr Pro Glu Val Thr Cys Val Val | |
| Val Asp Val Ser His Glu Asp Pro Glu Val Lys Phe Asn Trp Tyr Val Asp Gly Val Glu Val His Asn Ala | |
| Lys Thr Lys Pro Arg Glu Glu Gln Tyr Asn Ser Thr Tyr Arg Val Val Ser Val Leu Thr Val Leu His Gln | |
| Asp Trp Leu Asn Gly Lys Glu Tyr Lys Cys Lys Val Ser Asn Lys Ala Leu Pro Ala Pro Ile Glu Lys Thr | |
| Ile Ser Lys Ala Lys Gly Gln Pro Arg Glu Pro Gln Val Tyr Thr Leu Pro Pro Ser Arg Asp Glu Leu Thr | |
| Lys Asn Gln Val Ser Leu Thr Cys Leu Val Lys Gly Phe Tyr Pro Ser Asp Ile Ala Val Glu Trp Glu Ser | |
| Asn Gly Gln Pro Glu Asn Asn Tyr Lys Thr Thr Pro Pro Val Leu Asp Ser Asp Gly Ser Phe Phe Leu Tyr | |
| Ser Lys Leu Thr Val Asp Lys Ser Arg Trp Gln Gln Gly Asn Val Phe Ser Cys Ser Val Met His Glu Ala | |
| Leu His Asn His Tyr Thr Gln Lys Ser Leu Ser Leu Ser Pro Gly Lys | |
| Glu Pro Lys Ser Cys Asp Lys Thr His Thr Cys Pro Pro Cys Pro Ala Pro Glu Leu Leu Gly Gly Pro Ser | 29 |
| Val Phe Leu Phe Pro Pro Lys Pro Lys Asp Thr Leu Met Ile Ser Arg Thr Pro Glu Val Thr Cys Val Val | |
| Val Asp Val Ser His Glu Asp Pro Glu Val Lys Phe Asn Trp Tyr Val Asp Gly Val Glu Val His Asn Ala | |
| Lys Thr Lys Pro Arg Glu Glu Gln Tyr Asn Ser Thr Tyr Arg Val Val Ser Val Leu Thr Val Leu His Gln | |
| Asp Trp Leu Asn Gly Lys Glu Tyr Lys Cys Lys Val Ser Asn Lys Ala Leu Pro Ala Pro Ile Glu Lys Thr | |
| Ile Ser Lys Ala Lys Gly Gln Pro Arg Glu Pro Gln Val Tyr Thr Leu Pro Pro Ser Arg Glu Glu Met Thr | |
| Lys Asn Gln Val Ser Leu Thr Cys Leu Val Lys Gly Phe Tyr Pro Ser Asp Ile Ala Val Glu Trp Glu Ser | |
| Asn Gly Gln Pro Glu Asn Asn Tyr Lys Thr Thr Pro Pro Val Leu Asp Ser Asp Gly Ser Phe Phe Leu Tyr | |
| Ser Lys Leu Thr Val Asp Lys Ser Arg Trp Gln Gln Gly Asn Val Phe Ser Cys Ser Val Met His Glu Ala | |
| Leu His Asn His Tyr Thr Gln Lys Ser Leu Ser Leu Ser Pro Gly Lys | |
| Glu Leu Lys Thr Pro Leu Gly Asp Thr Thr His Thr Cys Pro Arg Cys Pro Glu Pro Lys Ser Cys Asp Thr | 30 |
| Pro Pro Pro Cys Pro Arg Cys Pro Glu Pro Lys Ser Cys Asp Thr Pro Pro Pro Cys Pro Arg Cys Pro Glu | |
| Pro Lys Ser Cys Asp Thr Pro Pro Pro Cys Pro Arg Cys Pro Ala Pro Glu Leu Leu Gly Gly Pro Ser Val | |
| Phe Leu Phe Pro Pro Lys Pro Lys Asp Thr Leu Met Ile Ser Arg Thr Pro Glu Val Thr Cys Val Val Val | |
| Asp Val Ser His Glu Asp Pro Glu Val Gln Phe Lys Trp Tyr Val Asp Gly Val Glu Val His Asn Ala Lys | |
| Thr Lys Pro Arg Glu Glu Gln Phe Asn Ser Thr Phe Arg Val Val Ser Val Leu Thr Val Leu His Gln Asp | |
| Trp Leu Asn Gly Lys Glu Tyr Lys Cys Lys Val Ser Asn Lys Ala Leu Pro Ala Pro Ile Glu Lys Thr Ile | |
| Ser Lys Thr Lys Gly Gln Pro Arg Glu Pro Gln Val Tyr Thr Leu Pro Pro Ser Arg Glu Glu Met Thr Lys | |
| Asn Gln Val Ser Leu Thr Cys Leu Val Lys Gly Phe Tyr Pro Ser Asp Ile Ala Val Glu Trp Glu Ser Ser | |
| Gly Gln Pro Glu Asn Asn Tyr Asn Thr Thr Pro Pro Met Leu Asp Ser Asp Gly Ser Phe Phe Leu Tyr Ser | |
| Lys Leu Thr Val Asp Lys Ser Arg Trp Gln Gln Gly Asn Ile Phe Ser Cys Ser Val Met His Glu Ala Leu | |
| His Asn Arg Phe Thr Gln Lys Ser Leu Ser Leu Ser Pro Gly Lys | |
| Glu Arg Lys Cys Cys Val Glu Cys Pro Pro Cys Pro Ala Pro Pro Val Ala Gly Pro Ser Val Phe Leu Phe | 31 |
| Pro Pro Lys Pro Lys Asp Thr Leu Met Ile Ser Arg Thr Pro Glu Val Thr Cys Val Val Val Asp Val Ser | |
| His Glu Asp Pro Glu Val Gln Phe Asn Trp Tyr Val Asp Gly Val Glu Val His Asn Ala Lys Thr Lys Pro | |
| Arg Glu Glu Gln Phe Asn Ser Thr Phe Arg Val Val Ser Val Leu Thr Val Val His Gln Asp Trp Leu Asn | |
| Gly Lys Glu Tyr Lys Cys Lys Val Ser Asn Lys Gly Leu Pro Ala Pro Ile Glu Lys Thr Ile Ser Lys Thr | |
| Lys Gly Gln Pro Arg Glu Pro Gln Val Tyr Thr Leu Pro Pro Ser Arg Glu Glu Met Thr Lys Asn Gln Val | |
| Ser Leu Thr Cys Leu Val Lys Gly Phe Tyr Pro Ser Asp Ile Ala Val Glu Trp Glu Ser Asn Gly Gln Pro | |
| Glu Asn Asn Tyr Lys Thr Thr Pro Pro Met Leu Asp Ser Asp Gly Ser Phe Phe Leu Tyr Ser Lys Leu Thr | |
| Val Asp Lys Ser Arg Trp Gln Gln Gly Asn Val Phe Ser Cys Ser Val Met His Glu Ala Leu His Asn His | |
| Tyr Thr Gln Lys Ser Leu Ser Leu Ser Pro Gly Lys | |
| Glu Ser Lys Tyr Gly Pro Pro Cys Pro Ser Cys Pro Ala Pro Glu Phe Leu Gly Gly Pro Ser Val Phe Leu | 32 |
| Phe Pro Pro Lys Pro Lys Asp Thr Leu Met Ile Ser Arg Thr Pro Glu Val Thr Cys Val Val Val Asp Val | |
| Ser Gln Glu Asp Pro Glu Val Gln Phe Asn Trp Tyr Val Asp Gly Val Glu Val His Asn Ala Lys Thr Lys | |
| Pro Arg Glu Glu Gln Phe Asn Ser Thr Tyr Arg Val Val Ser Val Leu Thr Val Leu His Gln Asp Trp Leu | |
| Asn Gly Lys Glu Tyr Lys Cys Lys Val Ser Asn Lys Gly Leu Pro Ser Ser Ile Glu Lys Thr Ile Ser Lys | |
| Ala Lys Gly Gln Pro Arg Glu Pro Gln Val Tyr Thr Leu Pro Pro Ser Gln Glu Glu Met Thr Lys Asn Gln | |
| Val Ser Leu Thr Cys Leu Val Lys Gly Phe Tyr Pro Ser Asp Ile Ala Val Glu Trp Glu Ser Asn Gly Gln | |
| Pro Glu Asn Asn Tyr Lys Thr Thr Pro Pro Val Leu Asp Ser Asp Gly Ser Phe Phe Leu Tyr Ser Arg Leu | |
| Thr Val Asp Lys Ser Arg Trp Gln Glu Gly Asn Val Phe Ser Cys Ser Val Met His Glu Ala Leu His Asn | |
| His Tyr Thr Gln Lys Ser Leu Ser Leu Ser Leu Gly Lys | |
| Glu Xaa Lys Ser Xaa Asp Xaa Thr Val Pro Cys Pro Xaa Cys Pro Ala Pro Glu Leu Leu Gly Gly Xaa | 33 |
| Xaa Xaa Xaa Xaa Xaa Xaa Xaa Xaa Xaa Xaa Xaa Pro Ser Val Phe Leu Phe Pro Pro Lys Pro Lys Asp | |
| Thr Leu Met Ile Ser Arg Thr Pro Glu Val Thr Cys Val Val Val Asp Val Ser His Glu Asp Pro Glu Val | |
| Xaa Phe Asn Trp Tyr Val Asp Gly Val Glu Val His Asn Ala Lys Thr Lys Pro Arg Glu Glu Gln Phe Asn | |
| Ser Thr Tyr Arg Val Val Ser Val Leu Thr Val Leu His Gln Asp Trp Leu Asn Gly Lys Glu Tyr Lys Cys | |
| Lys Val Ser Asn Lys Ala Leu Pro Ala Pro Ile Glu Lys Thr Ile Ser Lys Ala Lys Xaa Gly Gln Pro Arg | |
| Glu Pro Gln Val Tyr Thr Leu Pro Pro Xaa Ser Arg Glu Glu Met Thr Lys Asn Gln Val Ser Leu Thr Cys | |
| Leu Val Lys Gly Phe Tyr Pro Ser Asp Ile Ala Val Glu Trp Glu Ser Asn Gly Gln Xaa Xaa Pro Glu Asn | |
| Asn Tyr Lys Thr Thr Pro Pro Met Leu Asp Ser Asp Gly Xaa Xaa Xaa Ser Phe Phe Leu Tyr Ser Lys Leu | |
| Thr Val Asp Lys Ser Arg Trp Gln Gln Gly Asn Val Phe Ser Cys Ser Val Met His Glu Ala Leu His Asn | |
| His Tyr Thr Gln Lys Ser Leu Ser Leu Ser Pro Gly Lys Xaa Xaa | |

An Fc-Loop TPO-mimetic clone is transformed into E. coli by conventional methods known to those in the art. The isolated inclusion body fraction (1 g) is solubilized in 6 M guanidine-HCl, 50 mM Tris, 8 mM DTT, pH 9 (10 ml) at room temperature with mixing, for 1 hour. The denatured and reduced peptibody is refolded from the solubilized inclusion body fraction by a 1:25 (v/v) dilution into 2 M urea, 50 mM Tris, 4 mM cysteine, 1 mM cystamine, pH 8.5. The solubilized peptibody is added drop wise to the refold buffer at 4° C. with stirring. The refold reactions are allowed to stir for 48 hours, and then aliquots are evaluated by SDS-PAGE and reversed-phase HPLC.

Purification is achieved using a 2-column process. First a recombinant Protein-A column is equilibrated in 2 M urea, 50 mM Tris, pH 8.5 and loaded with the filtered peptibody refold reaction. The column is then washed with 2 column volumes of equilibration buffer, followed by 2 column volumes of PBS. The peptibody fraction is eluted with 50 mM NaOAc, pH3 and quickly neutralized by a 1:4 dilution into 10 mM NaOAc, 50 mM NaCl, pH 5. The diluted Protein-A eluate is again filtered and loaded to an SP Sepharose HP cation exchange column (Pharmacia) equilibrated in 10 mM NaOAc, 50 mM NaCl, pH 5. The peptibody fractions are then eluted with a linear 50-500 mM NaCl gradient, pooled and concentrated to about 2 mg/ml. The final pools of Fc-Loop TPO-mimetics are evaluated by SDS-PAGE and RP-HPLC. The final preparation of Fc-Loop TPO-mimetics are tested in an in vivo mouse bioassay.

Table 5 sets out the amino acid sequences of some TPO-mimetic peptides inserted into an Fc-Loop of SEQ ID NO: 24.

TABLE 5
TPO-MIMETIC PEPTIDES IN AN FC-LOOP

| AMINO ACID SEQUENCE | SEQ ID NO: |
|---|---|
| MDKTHTCPPCPAPELLGGPSVFLFPPKPKDTLMISRTPEVTCVVV | (FcL2) |
| DVSHEDPEVKFNWYVDGVEVHNAKTKPREEQYNSTYRVVSVLTVL | 44 |
| HQDWLNGKEYKCKVSNKALPAPIEKTISKAKGQPREPQVYTLPPS | |
| RDELGGQGCSSGGPTLREWQQCVQAQHSGGTKNQVSLTCLVKGFY | |
| PSDIAVEWESNGQPENNYKTTPPVLDSDGSFFLYSKLTVDKSRWQ | |
| QGNVFSCSVMHEALHNHYTQKSLSLSPGK | |
| MDKTHTCPPCPAPELLGGPSVFLFPPKPKDTLMISRTPEVTCVVV | (FcL3) |
| DVSHEDPEVKFNWYVDGVEVHNAKTKPREEQYNSTYRVVSVLTVL | 45 |
| HQDWLNGKEYKCKVSNKALPAPIEKTISKAKGQPREPQVYTLPPS | |
| RDELGGQGCSSGGPTLREWQQCVGAQHSGGTKNQVSLTCLVKGFY | |
| PSDIAVEWESNGQPENNYKTTPPVLDSDGSFFLYSKLTVDKSRWQ | |
| QGNVFSCSVMHEALHNHYTQKSLSLSPGK | |
| MDKTHTCPPCPAPELLGGPSVFLFPPKPKDTLMISRTPEVTCVVV | (FcL4) |
| DVSHEDPEVKFNWYVDGVEVHNAKTKPREEQYNSTYRVVSVLTVL | 46 |
| HQDWLNGKEYKCKVSNKALPAPIEKTISKAKGQPREPQVYTLPPS | |
| RDELGGQGCSSGGPTLREWQQCVHAQHS GGTKNQVSLTCLVKGF | |
| YPSDIAVEWESNGQPENNYKTTPPVLDSDGSFFLYSKLTVDKSRW | |
| QQGNVFSCSVMHEALHNHYTQKSLSLSPGK | |
| MDKTHTCPPCPAPELLGGPSVFLFPPKPKDTLMISRTPEVTCVVV | (FcL5) |
| DVSHEDPEVKFNWYVDGVEVHNAKTKPREEQYNSTYRVVSVLTVL | 47 |
| HQDWLNGKEYKCKVSNKALPAPIEKTISKAKGQPREPQVYTLPPS | |
| RDELGGQGCSSGGPTLREWQQCQGAQHSGGTKNQVSLTCLVKGFY | |
| PSDIAVEWESNGQPENNYKTTPPVLDSDGSFFLYSKLTVDKSRWQ | |
| QGNVFSCSVMHEALHNHYTQKSLSLSPGK | |
| MDKTHTCPPCPAPELLGGPSVFLFPPKPKDTLMISRTPEVTCVVV | (FcL6) |
| DVSHEDPEVKFNWYVDGVEVHNAKTKPREEQYNSTYRVVSVLTVL | 48 |
| HQDWLNGKEYKCKVSNKALPAPIEKTISKAKGQPREPQVYTLPPS | |
| RDELGGQGCSSGGPTLREWQQCVRPQHSGGTKNQVSLTCLVKGFY | |
| PSDIAVEWESNGQPENNYKTTPPVLDSDGSFFLYSKLTVDKSRWQ | |
| QGNVFSCSVMHEALHNHYTQKSLSLSPGK | |
| MDKTHTCPPCPAPELLGGPSVFLFPPKPKDTLMISRTPEVTCVVV | (FcL7) |
| DVSHEDPEVKFNWYVDGVEVHNAKTKPREEQYNSTYRVVSVLTVL | 49 |
| HQDWLNGKEYKCKVSNKALPAPIEKTISKAKGQPREPQVYTLPPS | |
| RDELGGQGCSSGGPTLREWQQCFRPQHSGGTKNQVSLTCLVKGFY | |
| PSDIAVEWESNGQPENNYKTTPPVLDSDGSFFLYSKLTVDKSRWQ | |
| QGNVFSCSVMHEALHNHYTQKSLSLSPGK | |
| MDKTHTCPPCPAPELLGGPSVFLFPPKPKDTLMISRTPEVTCVVV | (FcL8) |
| DVSHEDPEVKFNWYVDGVEVHNAKTKPREEQYNSTYRVVSVLTVL | 50 |
| HQDWLNGKEYKCKVSNKALPAPIEKTISKAKGQPREPQVYTLPPS | |
| RDELGGQGCSSGGPTLREWQQCFKAQHSGGTKNQVSLTCLVKGFY | |
| PSDIAVEWESNGQPENNYKTTPPVLDSDGSFFLYSKLTVDKSRWQ | |
| QGNVFSCSVMHEALHNHYTQKSLSLSPGK | |
| MDKTHTCPPCPAPELLGGPSVFLFPPKPKDTLMISRTPEVTCVVV | (FcL9) |
| DVSHEDPEVKFNWYVDGVEVHNAKTKPREEQYNSTYRVVSVLTVL | 51 |
| HQDWLNGKEYKCKVSNKALPAPIEKTISKAKGQPREPQVYTLPPS | |
| RDELGGQGCSSGGPTLREWQQCVKPQHSGGTKNQVSLTCLVKGFY | |
| PSDIAVEWESNGQPENNYKTTPPVLDSDGSFFLYSKLTVDKSRWQ | |
| QGNVFSCSVMHEALHNHYTQKSLSLSPGK | |
| MDKTHTCPPCPAPELLGGPSVFLFPPKPKDTLMISRTPEVTCVVV | (FcL10) |
| DVSHEDPEVKFNWYVDGVEVHNAKTKPREEQYNSTYRVVSVLTVL | 52 |
| HQDWLNGKEYKCKVSNKALPAPIEKTISKAKGQPREPQVYTLPPS | |
| RDELGGQGCSSGGPTLREWQQCVRAQHSGGTKNQVSLTCLVKGFY | |
| PSDIAVEWESNGQPENNYKTTPPVLDSDGSFFLYSKLTVDKSRWQ | |
| QGNVFSCSVMHEALHNHYTQKSLSLSPGK | |
| MDKTHTCPPCPAPELLGGPSVFLFPPKPKDTLMISRTPEVTCVVV | (FcL11) |
| DVSHEDPEVKFNWYVDGVEVHNAKTKPREEQYNSTYRVVSVLTVL | 53 |
| HQDWLNGKEYKCKVSNKALPAPIEKTISKAKGQPREPQVYTLPPS | |
| RDELGGQGCSSGGPTLREWQQCRPAQHSGGTKNQVSLTCLVKGFY | |
| PSDIAVEWESNGQPENNYKTTPPVLDSDGSFFLYSKLTVDKSRWQ | |
| QGNVFSCSVMHEALHNHYTQKSLSLSPGK | |
| MDKTHTCPPCPAPELLGGPSVFLFPPKPKDTLMISRTPEVTCVVV | (FcL12) |
| DVSHEDPEVKFNWYVDGVEVHNAKTKPREEQYNSTYRVVSVLTVL | 54 |
| HQDWLNGKEYKCKVSNKALPAPIEKTISKAKGQPREPQVYTLPPS | |
| RDELGG QGCSSGGPTLREWQQCRRPQHS GGTKNQVSLTCLVKG | |
| FYPSDIAVEWESNGQPENNYKTTPPVLDSDGSFFLYSKLTVDKSR | |
| WQQGNVFSCSVMHEALHNHYTQKSLSLSPGK | |
| MDKTHTCPPCPAPELLGGPSVFLFPPKPKDTLMISRTPEVTCVVV | (FcL13) |
| DVSHEDPEVKFNWYVDGVEVHNAKTKPREEQYNSTYRVVSVLTVL | 55 |
| HQDWLNGKEYKCKVSNKALPAPIEKTISKAKGQPREPQVYTLPPS | |
| RDELGGQGCSSGGPTLREWQQCQRAQHSGGTKNQVSLTCLVKGFY | |
| PSDIAVEWESNGQPENNYKTTPPVLDSDGSFFLYSKLTVDKSRWQ | |
| QGNVFSCSVMHEALHNHYTQKSLSLSPGK | |
| MDKTHTCPPCPAPELLGGPSVFLFPPKPKDTLMISRTPEVTCVVV | (FcL14) |
| DVSHEDPEVKFNWYVDGVEVHNAKTKPREEQYNSTYRVVSVLTVL | 56 |
| HQDWLNGKEYKCKVSNKALPAPIEKTISKAKGQPREPQVYTLPPS | |
| RDELGGQGCSSGGPTLREWQQCSRAQHSGGTKNQVSLTCLVKGFY | |
| PSDIAVEWESNGQPENNYKTTPPVLDSDGSFFLYSKLTVDKSRWQ | |
| QGNVFSCSVMHEALHNHYTQKSLSLSPGK | |

There is a high degree of homology in the secondary and tertiary structural conformations within the Fc domains of different IgG subtypes and between species. The x-ray crystal structure coordinates for these structures can be found in the RCSB Protein Data Bank (http://www.rcsb.org/pdb/).

In the human IgG1 Fc sequence (SEQ ID NO: 24) used for peptibody fusions, predicted Fc-Loop regions are found in SEQ ID NOS: 57, 58, 59, 60, 61, 62, 63, 64, 65, and 66. Any, or all of these sites may be suitable for full or partial replacement by or insertion of peptide sequences and are considered part of this invention. Specifically preferred internal sites are SEQ ID NOS: 67, 68, 69, 70, 71, 72, and 73. One preferred site is SEQ ID NO: 70, between Leuᵢ₃₉ and Thr₁₄₀ in the DELTK (SEQ ID NO: 63) loop into which peptide FcL2-FcL14 have been instered as set forth in Table 5. Potential loop sites in other Ig subtypes are understood in the art.

Exemplary amino acid sequences of human Fc regions from IgA, IgM and IgG subtypes are SEQ ID NOS: 25 to 32). A consensus sequence is provided in SEQ ID NO: 33.

Preferred internal sites for peptide addition that correspond to those of the Fc sequence in SEQ ID NO: 3 are set out as follows:

SEQ ID NO: 57 within SEQ ID NOS: 28 to 33;

SEQ ID NO: 58 within SEQ ID NOS: 28 to 31 and 33;

SEQ ID NO: 74 within SEQ ID NO: 33;

SEQ ID NO: 59 within SEQ ID NO: 28 to 33;

SEQ ID NO: 60 within SEQ ID NOS: 28 and 29;

SEQ ID NO: 74 within SEQ ID NOS: 30 to 33;

SEQ ID NO: 61 within SEQ ID NOS: 28 to 30, 32, and 33;

SEQ ID NO: 76 within SEQ ID NO: 31;

SEQ ID NO: 62 within SEQ ID NOS: 28, 29, and 33;

SEQ ID NO: 77 within SEQ ID NO: 30;

SEQ ID NO: 78 within SEQ ID NO: 31;

SEQ ID NO: 79 within SEQ ID NO: 32;

SEQ ID NO: 63 within SEQ ID NO: 347;

SEQ ID NO: 80 within SEQ ID NOS: 29 to 33;

SEQ ID NO: 64 within SEQ ID NOS: 28, 29, 31, 32, and 33;

SEQ ID NO: 81 within SEQ ID NO: 30;

SEQ ID NO: 65 within SEQ ID NOS: 28, 29, and 32;

SEQ ID NO: 82 within SEQ ID NOS: 30, 31 and 33;

SEQ ID NO: 66 within SEQ ID NOS: 28, 29, 31, and 33;

SEQ ID NO: 83 within SEQ ID NO: 30; and

SEQ ID NO: 84 within SEQ ID NO: 32.

Sequence alignments suggest two more potential insertion sites at Q167/P168 and/or G183/5184 (using the numbering of SEQ ID NO: 3). These positions correspond to gaps in the IgG sequences where there are two and three residue insertions found in the aligned IgA and IgM sequences. Some preferred insertion sites are set out as follows:

H₅₃/E₅₄ in SEQ ID NOS: 28 and 29;

H₁₀₀/E₁₀₁ in SEQ ID NO: 30;

H₄₉/E₅₀ in SEQ ID NO: 31;

Q₅₀/E₅₁ in SEQ ID NO: 32;

H₁₁₂/E₁₁₃ in SEQ ID NO: 33;

Y₈₁/N₈₂ in SEQ ID NOS: 28 and 29;

F₁₂₈/N₁₂₉ in SEQ ID NO: 30;

F₇₇/N₇₈ in SEQ ID NO: 31;

F₇₈/N₇₉ in SEQ ID NO: 32;

F₁₄₀/N₁₄₁ in SEQ ID NO: 33;

N₁₁₀/K₁₁₁ in SEQ ID NOS: 28 and 29;

N₁₅₇/K₁₅₈ in SEQ ID NO: 30;

N₁₀₆/K₁₁₀₇ in SEQ ID NO: 31;

N₁₀₇/K₁₀₈ in SEQ ID NO: 32;

N₁₆₉/K₁₇₀ in SEQ ID NO: 33;

L₁₄₃/T₁₄₄ in SEQ ID NOS: 28 and 29;

M₁₉₀/T₁₉₁ in SEQ ID NO: 30;

M₁₃₉/T₁₄₀ in SEQ ID NO: 31;

M₁₄₀/T₁₄₁ in SEQ ID NO: 32;

M₂₀₄/T₂₀₅ in SEQ ID NO: 33;

Q₁₇₁/P₁₇₂ in SEQ ID NOS: 28 and 29;

Q₂₁₈/P₂₁₉ in SEQ ID NO: 30;

Q₁₆₇/P₁₆₈ in SEQ ID NO: 31;

Q₁₆₈/P₁₆₉ in SEQ ID NO: 32;

Q₂₃₂/P₂₃₃ in SEQ ID NO: 33;

E₁₇₃/N₁₇₄ in SEQ ID NOS: 28 and 29;

E₂₂₀/N₂₂₁ in SEQ ID NO: 30;

E₁₆₉/P₁₇₀ in SEQ ID NO: 31;

E₁₇₀/N₁₇₁ in SEQ ID NO: 32;

E₂₃₄/N₂₃₅ in SEQ ID NO: 33;

S₁₈₆/D₁₈₇ in SEQ ID NOS: 28 and 29;

S₂₃₂/D₂₃₃ in SEQ ID NO: 30;

S₁₈₁/D₁₈₂ in SEQ ID NO: 31;

S₁₈₂/D₁₈₃ in SEQ ID NO: 32;

S₂₄₆/D₂₄₇ in SEQ ID NO: 33;

G₁₈₈/S₁₈₉ in SEQ ID NOS: 28 and 29;

G₂₃₄/S₂₃₅ in SEQ ID NO: 30;

G₁₈₃/S₁₈₄ in SEQ ID NO: 31;

G₁₈₄/S₁₈₅ in SEQ ID NO: 32;

G₂₄₈/S₂₄₉ in SEQ ID NO: 33;

G₂₀₅/N₂₀₆ in SEQ ID NOS: 28 and 29;

G₂₅₂/N₂₅₃ in SEQ ID NO: 30;

G₂₀₁/N₂₀₂ in SEQ ID NO: 31;

G₂₀₂/N₂₀₃ in SEQ ID NO: 32; and

G₂₆₈/N₂₆₉ in SEQ ID NO: 33.

An alignment of human IgG1 Fc domain (SEQ ID NO: 85) used for the peptibody platform with rat IgG2A from crystal structure of FcRn/Fc complex (SEQ ID NO: 86) provided a consensus sequence (SEQ ID NO: 87).

Table 6 sets out amino acid sequences of some of the Fc sequences for use in the present invention and some of the internal sites for peptide addition/insertion.

TABLE 6
AMINO ACID SEQUENCES OF IGG SEQUENCES AND INSERTION SITES

| AMINO ACID SEQUENCE | SEQ ID NO: |
|---|---|
| Glu Gly Gly Gly Gly Gly Asp Lys Thr His Thr Cys Pro Pro Cys Pro | 85 |
| Ala Pro Glu Leu Leu Gly Gly Pro Ser Val Phe Leu Phe Pro Pro Lys | |
| Pro Lys Asp Thr Leu Met Ile Ser Arg Thr Pro Glu Val Thr Cys Val | |
| Val Val Asp Val Ser His Glu Asp Pro Glu Val Lys Phe Asn Trp Tyr | |
| Val Asp Gly Val Glu Val His Asn Ala Lys Thr Lys Pro Arg Glu Glu | |
| Gln Tyr Asn Ser Thr Tyr Arg Val Val Ser Val Leu Thr Val Leu His | |
| Gln Asp Trp Leu Asn Gly Lys Glu Tyr Lys Cys Lys Val Ser Asn Lys | |
| Ala Leu Pro Ala Pro Ile Glu Lys Thr Ile Ser Lys Ala Lys Gly Gln | |
| Pro Arg Glu Pro Gln Val Tyr Thr Leu Pro Pro Ser Arg Asp Glu Leu | |
| Thr Lys Asn Gln Val Ser Ser Arg Trp Gln Gln Gly Asn Val Phe Ser | |
| Cys Ser Val Met His Glu Ala Leu His Asn His Tyr Thr Gln Lys Ser | |
| Leu Ser Leu Ser Pro Gly Lys | |
| Ser Val Phe Ile Phe Pro Pro Lys Thr Lys Asp Val Leu Thr Ile Thr | 86 |
| Leu Thr Pro Lys Val Thr Cys Val Val Val Asp Ile Ser Gln Asn Asp | |
| Pro Glu Val Arg Phe Ser Trp Phe Ile Asp Asp Val Glu Val His Thr | |
| Ala Gln Thr His Ala Pro Glu Lys Gln Ser Asn Ser Thr Leu Arg Ser | |
| Val Ser Glu Leu Pro Ile Val His Arg Asp Trp Leu Asn Gly Lys Thr | |
| Phe Lys Cys Lys Val Asn Ser Gly Ala Phe Pro Ala Pro Ile Glu Lys | |
| Ser Ile Ser Lys Pro Glu Gly Thr Pro Arg Gly Pro Gln Val Tyr Thr | |
| Met Ala Pro Pro Lys Glu Glu Met Thr Gln Ser Gln Val Ser Ile Thr | |
| Cys Met Val Lys Gly Phe Tyr Pro Pro Asp Ile Tyr Thr Glu Trp Lys | |
| Met Asn Gly Gln Pro Gln Glu Asn Tyr Lys Asn Thr Pro Pro Thr Met | |
| Asp Thr Asp Gly Ser Tyr Phe Leu Tyr Ser Lys Leu Asn Val Lys Lys | |
| Glu Thr Trp Gln Gln Gly Asn Thr Phe Thr Cys Ser Val Leu His Glu | |
| Gly Leu His Asn His His Thr Glu Lys Ser Leu Ser His | |
| Ser Val Phe Ile Phe Pro Pro Lys Xaa Lys Asp Xaa Leu Xaa Ile Ser | 87 |
| Xaa Thr Pro Xaa Val Thr Cys Val Val Val Asp Ile Ser Xaa Xaa Asp | |
| Pro Glu Val Lys Phe Xaa Trp Phe Ile Asp Xaa Val Glu Val His Xaa | |
| Ala Xaa Thr Xaa Xaa Xaa Glu Xaa Gln Xaa Asn Ser Thr Xaa Arg Xaa | |
| Val Ser Xaa Leu Ile Leu His Xaa Asp Trp Leu Asn Gly Lys Xaa Phe | |
| Lys Cys Lys Val Xaa Xaa Xaa Ala Xaa Pro Ala Pro Ile Glu Lys Ser | |
| Ile Ser Lys Xaa Xaa Gly Xaa Pro Arg Xaa Pro Gln Val Tyr Thr Leu | |
| Xaa Pro Xaa Lys Asp Glu Leu Thr Xaa Xaa Gln Val Ser Ile Thr Cys | |
| Leu Val Lys Gly Phe Tyr Pro Xaa Asp Ile Xaa Xaa Glu Trp Xaa Xaa | |
| Asn Gly Gln Pro Xaa Xaa Asn Tyr Lys Xaa Thr Pro Pro Xaa Leu Asp | |
| Ser Asp Gly Ser Phe Phe Leu Tyr Ser Lys Leu Xaa Val Xaa Lys Xaa | |
| Xaa Trp Gln Gln Gly Asn Xaa Phe Ser Cys Ser Val Leu His Glu Ala | |
| Leu His Asn His His Thr Xaa Lys Ser Leu Ser Xaa | |
| Lys Ser Arg Trp Gln Gln Gly Asn Ile | 83 |
| Lys Ser Arg Trp Gln Glu Gly Asn Val | 84 |
| Pro Pro | 57 |
| Asp Val Ser His Glu Asp Pro Glu | 58 |
| Ser His Glu | 67 |
| Val His Asn Ala | 59 |
| Glu Glu Gln Tyr Asn Ser Thr | 60 |
| Tyr Asn Ser | 68 |
| Val Leu His Gln Asp Trp Leu Asn Gly Lys Glu | 61 |
| Asn Lys Ala Leu Pro Ala Pro Ile Glu Lys Thr Ile Ser Lys Ala Lys | 62 |
| Asn Lys Ala | 69 |
| Asp Glu Leu Thr Lys | 63 |
| Leu Thr Lys | 70 |
| Asn Gly Gln Pro Glu Asn Asn | 64 |
| Glu Asn Asn | 71 |
| Thr Thr Pro Pro Val Leu Asp Ser Asp Gly Ser | 65 |
| Val Leu Asp Ser Asp | 72 |
| Lys Ser Arg Trp Gln Gln Gly Asn Val | 66 |
| Gln Gly Asn | 73 |
| Asp Val Ser Gln Glu Asp Pro Glu | 74 |
| Glu Glu Gln Phe Asn Ser Thr | 75 |
| Val Val His Gln Asp Trp Leu Asn Gly Lys Glu | 76 |
| Asn Lys Ala Leu Pro Ala Pro Ile Glu Lys Thr Ile Ser Lys Thr Lys | 77 |
| Gly Gln Pro Arg Glu Pro | |
| Asn Lys Gly Leu Pro Ala Pro Ile Glu Lys Thr Ile Ser Lys Thr Lys | 78 |
| Gly Gln Pro Arg Glu Pro | |
| Asn Lys Gly Leu Pro Ser Ser Ile Glu Lys Ala Lys Gly Gln Pro Arg | 79 |
| Glu Pro | |
| Glu Glu Met Thr Lys | 80 |
| Ser Gly Gln Pro Glu Asn Asn | 81 |
| Thr Thr Pro Pro Met Leu Asp Ser Asp Gly Ser | 82 |

Table 7 sets out still other TPO-mimetic peptides having c-mpl receptor binding activity. These peptides are contemplated for use alone or as TPO-mimetic fusion proteins, wherein the TPO-mimetic peptide is fused to either an N-terminus of an Fc region or within an Fc-Loop, a modified Fc molecule. Fc-Loops are described herein and in U.S. Patent Application Publication No. US2006/0140934 incorporated herein by reference in its entirety.

TABLE 7
TPO-MIMETIC PEPTIDES

| AMINO ACID SEQUENCE | SEQ ID NO: |
|---|---|
| CSSGGPTLREWQQCSRAQ | 8 |
| CSSGGPTLREWQQCQRAQ | 9 |
| CSSGGPTLREWQQCGRAQ | 10 |

Table 8 reports the effective concentration (Pb EC50 in ng/ml) at which some of the TPO-mimetic fusion proteins of the invention demonstrate peptibody activity based on an in vitro activity assay using murine 32D cells expressing human MPL in a reporter assay format as described herein above. This TPO in vitro bioassay is a mitogenic assay utilizing an IL-3 dependent clone of murine 32D cells that have been transfected with human mpl receptor. This assay is described in greater detail in WO 95/26746. An Fc molecule is fused at either the N-terminus or the C-terminus of the peptide. Some TPO-mimetics in this table were inserted into an Fc-Loop, comprise an Fc molecule connected at the N-terminus of two different peptide sequences connected in tandem, or comprise an Fc molecule connected at the N-terminus of two tandem copies of the same peptide.

TABLE 8
ACTIVITY OF SOME TPO-MIMETIC PEPTIDES

| | | Pb EC50 |
|---|---|---|
| TPO-Mimetic | Peptide Sequences Used in the TPO-Mimetic | (ng/ml) |
| Fc-Loop- | CSSGGPTLREWQQCSRAQ | 0.28 |
| (SEQ ID NO: 8) | (SEQ ID NO: 8) | |
| Fc-Loop- | CSSGGPTLREWQQCQRAQ | 0.27 |
| (SEQ ID NO: 9) | (SEQ ID NO: 9) | |
| Fc-Loop- | CSSGGPTLREWQQCGRAQ | 2.31 |
| (SEQ ID NO: 10) | (SEQ ID NO: 10) | |
| SEQ ID NO: 35 | CSSGGPTLREWQQCRRMQ | 0.44 |
| | (SEQ ID NO: 35) | |
| SEQ ID NO: 37 | CSWGGPTLKNWLQCVRAK | 4.01 |
| | (SEQ ID NO: 37) | |

Table 9 reports the in vitro and in vivo activity of some TPO-mimetic compounds of the invention. The constructs set out in Table 9 were assessed for in vitro activity using murine 32D cells expressing human MPL in a reporter assay format as described herein above. This TPO in vitro bioassay is a mitogenic assay utilizing an IL-3 dependent clone of murine 32D cells that have been transfected with human mpl receptor. This assay is described in greater detail in WO 95/26746. The activity of the constructs was determined to be comparable when considering reasonable assay variance.

The constructs were also subjected to an in vivo activity study by by injecting mice with 3, 5, 50, 100, or 200 μg/kg of the noted construct and then observing the change in platelet number over a 17-day period. An in vivo activity of “++++” indicates high activity, while an in vivo activity of “+” denotes low activity. Using this in vivo assay system, all eight TPO-mimetic compounds shown in Table 9 appeared to be indistinguishable.

Fc-(SEQ ID NO: 35) (M19A) indicates that the M at amino acid position 19 in SEQ ID NO: 35 is replaced with an A. Fc-(SEQ ID NO: 35) (R17V) indicates that the R at amino acid position 17 is replaced with a V. Accordingly, Fc-(SEQ ID NO: 35) (R17V/M19A) denotes that there are two substitutions in SEQ ID NO: 35; the R at position 17 is replaced with a V and the M at position 19 is replaced with an A. Fc-Loop (Asym) (SEQ ID NO: 35) in Table 9 denotes that SEQ ID NO: 35 is inserted into the loop region of the Fc at position L139/T140 using four glycine spacers at the N-terminus and two glycine spacers at the C-terminus. Fc-Loop (Sym) (SEQ ID NO: 35) in Table 9 denotes that SEQ ID NO: 35 is inserted into the loop region of the Fc at position L139/T140 using two glycine spacers at both the N- and C-termini.

TABLE 9
TPO-MIMETIC FUSION PROTEIN ACTIVITY
IN VITRO AND IN VIVO

| | In vitro | In vitro | |
|---|---|---|---|
| Construct | EC₅₀ (pM) | EC₅₀ (95% CI) | In vivo |
| Fc-(SEQ ID NO: 35) | 14.6 | 8.9-24.0 | ++++ |
| Fc-(SEQ ID NO: 35) | 10.4 | 8.0-13.6 | ++++ |
| (M19A) | | | |
| Fc-(SEQ ID NO: 35) | 25.1 | 14.0-45.2 | ++++ |
| (R17V) | | | |
| Fc-(SEQ ID NO: | 5.5 | 3.6-8.4 | ++++ |
| 35)(R17V/M19A) | | | |
| Fc-Loop(Asym) | 12.7 | 9.1-17.7 | ++++ |
| (SEQ ID NO: 35) | | | |
| Fc-Loop(Sym) | 13.7 | 10.1-18.5 | ++++ |
| (SEQ ID NO: 35) | | | |
| Fc-Loop(Asym-R17V) | 5.7 | 4.2-7.7 | ++++ |
| Fc-Loop(Sym-R17V) | 9.9 | 6.9-14.0 | ++++ |

Table 10 further reports the in vitro activity of some TPO-mimetic compounds of the invention. Fc-Loop (Asym) (SEQ ID NO: 35) in Table 10 denotes that SEQ ID NO: 35 was inserted into the loop region of the Fc at position L139/T140 using four glycine spacers at the N-terminus. Fc-Loop (Asym) (SEQ ID NO: 5) in Table 10 denotes that SEQ ID NO: 5 was inserted into the loop region of the Fc at position L139/T140 using four glycine spacers at the N-terminus and two glycine spacers at the C-terminus. The appended “-C” at the end of the construct name in Table 10 denotes that the purified cyclic form (the cysteines in SEQ ID NO: 35 form an intrachain disulfide bond). The appended “XL” at the end of the construct name in Table 10 denotes that the purified cross-linked form (the cysteines in SEQ ID NO: 35 form an interchain disulfide bond). The appended “-Mixed” at the end of the construct name in Table 10 denotes that there is a mixture of the cyclic and cross-linked forms. Fc-Loop (Sym) (SEQ ID NO: 35 or 5 or 6) in Table 10 denotes that SEQ ID NO: 35, 5, or 6 was inserted into the loop region of the Fc at position L139/T140 using two glycine spacers at the N-terminus and two glycine spacers at the C-terminus.

TPO-dependent proliferation of 32Dcl23/Mpl cells and differentiation of primary human CD34+ progenitors were used to measure the in vitro potency of TPO-mimetic compounds. In the latter assay, the percentage of cells expressing the CD61 surface marker was chosen as the key parameter to measure megakaryocytic differentiation. For both assays, measurements were expressed as POC relative to the peak value (cell proliferation or differentiation) measured for a well-characterized positive control. At least three determinations for each molecule were performed in the 32Dcl23/Mpl proliferation assay, and at least three determinations on two separate donors were performed for each molecule in the CD34+ differentiation assay.

### CD34+ Liquid Culture Assay

StemPro-34 Serum-Free Media supplemented with 100 ng/mL recombinant human Stem Cell Factor (rhSCF, Amgen, Inc.) was used as the growth medium. CD34+ cells were obtained from normal, G-CSF mobilized donors, provided by All Cells, Inc. All experiments were performed in 96-well plates using 5−20×10³ CD34+ cells/well.

Two solutions of each TPO-mimetic compound (or peptibody) were prepared at a concentration of 2 μg/mL and 0.6 μg/mL, respectively. From each of these solutions, 1:10 serial dilutions were made into a 96-well tissue culture plate containing a volume of 180 μl/well (20 μl of sample into 200 μl final) of growth medium to obtain a concentration curve of 200, 60, 20, 6, 2, 0.6, and 0.2 ng/mL. Next, 100 μl from each well was transferred into another 96-well plate and 100 μl (5-20,000 CD34+ cells) of cells resuspended in SP34 media (supplemented with 100 ng/mL SCF) were added. The final concentration of the test molecules was 100, 30, 10, 3, 1.0, 0.3 and 0.1 ng/mL.

The tissue culture plate was cultured in 5% CO₂ in 100% humidified air at 37° C. for 7 days. Next, the cells were stained in the 96-well plate (per BD Biosciences protocol) with 2 μl (0.1 μg)/well FITC-CD15 or 0.5 μl (0.1 μg)/well APC-CD61 along with the appropriate isotype controls. Just before analysis, 1 μl (0.05 μg) of propidium iodide was added to each well, to stain dead cells. Live cells were identified by appropriate FSC/SSC gating and propidium iodide exclusion. Data were acquired on a FACSCalibur flow cytometer (Beckton Dickinson).

### 32Dcl23/Mpl Cell Proliferation Assay

32Dcl23/Mpl cells were cultured at 37° C. in 5% CO₂, in MEM containing 10% FBS, PGS (100 units/mL penicillin G sodium, 100 μg/mL streptomycin sulfate, 292 μg/mL L-glutamine), and 5 ng/mL murine IL-3. Cell viability greater than 80% was confirmed by the Beckman Coulter Vi-Cell XR instrument (Beckman Coulter Inc., Fullerton, Calif.).

For each experiment, 32Dcl23/Mpl cells were washed twice in growth medium, and the cells pellet was resuspended in 1×10⁶ cells per mL. Cells were plated in 96-well Costar round bottom plates at a cell density of 60,000 cells per well (60 μL per well).

Test molecules were serially diluted 1:3 in growth medium, to obtain a dose range from 40 ng/mL to 0.01 ng/mL. Sixty microliters of the diluted peptibody were added to the cell plate containing 60 μL of 60,000 cells per well. The treated cells were incubated for 24 hours in 5% CO₂ humidifier incubator. Cellular ATP was then measured as surrogate marker for cell proliferation with the Promega CellTiter-Glo reagent (Cat # G7572), according to the manufacturer's specifications. Luminescence signal was measured with Molecular Devices LMax³⁸⁴ instrument (Molecular Devices Inc., Sunnyvale, Calif.).

### Data Analysis

Percentages of cells expressing CD61 were calculated with the FCS Express v3.0 software, gating for live cells based on forward scatter/side scatter and propidium iodide exclusion. Dose responses were plotted with Spotfire DecisionSite v8.2.1.

### Statistical Analysis

Data were plotted as mean±SD. For relevant candidates, EC₅₀s were calculated with the GraphPad Prism v4.01 software package using the following sigmoidal dose-response equation:

Y=min+(max−min)/(1+10̂((Log EC50-X)))

where X is the logarithm of concentration, and Y the response.

TABLE 10
TPO-MIMETIC FUSION PROTEIN ACTIVITY

| | EC₅₀ on 32Dcl23/Mpl | |
|---|---|---|
| Construct Sequence | (pM) | EC₅₀ on CD34+ (pM) |
| Fc-Loop (Asym) | 13 | 0.1 |
| (SEQ ID NO: 35) | | |
| Fc-Loop (Sym) | 15 | 2 |
| (SEQ ID NO: 35) | | |
| Fc-Loop (Asym) | 6 | 5 |
| (SEQ ID NO: 5) | | |
| Fc-(SEQ ID NO: 35) | 10 | 6 |
| (M19A) | | |
| Fc-(SEQ ID NO: 35) | 5 | 6 |
| (R17V/M19A)-C | | |
| Fc-(SEQ ID NO: 35) | 9 | 5 |
| (R17V/M19A)-XL | | |
| Fc-(SEQ ID NO: 35) | 15 | 5 |
| (R17V)-XL | | |
| Fc-(SEQ ID NO: 35) | 11 | 0.01 |
| (R17V)-Mixed | | |
| Fc-(SEQ ID NO: 35) | 25 | 3 |
| (R17V) | | |
| Fc-(SEQ ID NO: 35) | 15 | 5 |
| Fc-Loop (Sym) | 10 | 5 |
| (SEQ ID NO: 5) | | |
| Fc-Loop (Sym) | 12 | 0.6 |
| (SEQ ID NO: 6) | | |

Some of the TPO-mimetics set out above also have been tested directly for biological activity in vivo in mice and results showed that some TPO-mimetic peptides and TPO-mimetic fusion proteins are more effective than others in stimulating platelet production in mice.

### Mutations to Decrease Protease Sensitivity

Experimental results showed that certain TMP constructs were susceptible to protease cleavage. In an attempt to reduce loss of bioactivity associated with this type of degradation, a series of modifications were introduced in the amino acid sequence of the TMP set out in SEQ ID NO: 88 which were then assessed for changes in activity compared to the parent TMP sequence.

(SEQ ID NO: 88)
MDKTHTCPPCPAPELLGGPSVFLFPPKPKDTLMISRTPEVTCVVVDV
SHEDPEVKFNWYVDGVEVHNAKTKPREEQYNSTRYVVSVLTVLHQDW
LNGKEYKCKVSNKALPAPIEKTISKAKGQPREPQVYTLPPSRDELGG
QGCSSGGPTLREWQQCRRAQHSGGTKNQVSLTCLVKGFYPSDIAVEW
ESNGQPENNYKTTPPVLDSDGSFFLYSKLTVDKSRWQQGNVFSCSVM
HELALHNH

The wild type parent sequence set out in SEQ ID NO: 88 (FcL1) was mutated as indicated below in Table 11. The mutants are defined by amino acid changes at specific residues wherein numbering begins in the underlined region corresponding to SEQ ID NO:6 of the wild type construct FcL1. Specific mutations were introduced at the residues indicated in bold (i.e., RRA).

TABLE 11

| TPO MIMETIC | Mutations | SEQ ID NO: |
|---|---|---|
| FcL1 | Wild Type | 6 |
| FcL2 | R17V/R18Q | 11 |
| FcL3 | R17V/R18G | 12 |
| FcL4 | R17V/R18H | 13 |
| FcL5 | R17Q/R18G | 14 |
| FcL6 | R17V/A19P | 15 |
| FcL7 | R17F/A19P | 16 |
| FcL8 | R17F/R18K | 17 |
| FcL9 | R17V/R18K/A19P | 18 |
| FcL10 | R17V | 19 |
| FcL11 | R18P | 20 |
| FcL12 | A19P | 21 |
| FcL13 | R17Q | 22 |
| FcL14 | R17S | 23 |

In vitro cell proliferation activity experiments indicated that a number of the mutants set out in Table 11 displayed activity comparable to that of the wild type construct. In protease sensitivity assays, constructs designated FcL2, FcL3, FcL4, FcL7, and FcL8 (corresponding to SEQ ID NO: 11, 12, 13, 16 and 17 respectively) were found to be more resistant to proteolysis than the wild type construct FcL1 (SEQ ID NO:6), with the FcL2, FcL3, and FcL4 (corresponding to SEQ ID NO: 11, 12 and 13 respectively) mutants being the most resistant to proteolytic cleavage.

In addition to the TPO mimetic compounds set forth in Table 11 as SEQ ID NOs:11-23, also contemplated herein are TPO mimetics comprising various combinations of the mutations introduced to FcL1 (SEQ ID NO:6). For example, contemplated herein is a TPO mimetic peptide having the mutations of FcL10, FcL11, FcL12 introduced into SEQ ID NO:6, such that the amino acids V-P-P occur at positions 17, 18 and 19 relative to SEQ ID NO:6. Another example of a combination contemplated herein is the mutations of FcL13, FcL11, FcL12 introduced into SEQ ID NO:6, such that the amino acids Q-P-P occur at positions 17, 18 and 19 relative to SEQ ID NO:6. Another example of a combination contemplated herein is the mutation of either one of FcL2, FcL3 or FcL4 in combination with FcL12 introduced into SEQ ID NO:6, such that the amino acids V-Q-P (FcL2/Fc12 combo), V-G-P (FcL3/Fc12 combo), or V-H-P (FcL4/Fc12 combo), respectively, occur at positions 17, 18 and 19 relative to SEQ ID NO:6. Another example of a combination contemplated herein is the mutation of FcL8 in combination with FcL12 introduced into SEQ ID NO:6, such that the amino acids F-K-P (FcL8/Fc12 combo) occur at positions 17, 18 and 19 relative to SEQ ID NO:6; and the like.

The invention now being fully described, it will be apparent to one of ordinary skill in the art that many changes and modifications can be made thereto, without departing from the spirit and scope of the invention as set forth herein.

We claim:

1. A compound that binds to an mpl receptor comprising a structure set out in Formula I,
[(X¹)ₐ-(F¹)_{z}-(X²)_{b}]-(L¹)_{c}-WSP_{d} Formula I
and multimers thereof, wherein:
F¹ is a vehicle;
X¹ is independently selected from:
P¹-(L²)ₑ-
P²-(L³)_{f}-P¹-(L²)ₑ-
P³-(L⁴)_{g}-P²-(L³)_{f}-P¹-(L²)ₑ- and
P⁴-(L⁵)ₕ-P³-(L⁴)_{g}-P²-(L³)_{f}-P¹-(L²)ₑ-
X² is independently selected from:
-(L²)ₑ-P¹,
-(L²)ₑ-P¹-(L³)_{f}-P²,
-(L²)ₑ-P¹-(L³)_{f}-P²-(L⁴)_{g}-P³, and
-(L²)ₑ-P¹-(L³)_{f}-P²-(L⁴)_{g}-P³-(L⁵)ₕ-P⁴
wherein P¹, P², P³, and P⁴ are each independently sequences of pharmacologically active peptides;
L¹, L², L³, L⁴, and L⁵ are each independently linkers;
a, b, c, d, e, f, g, and h are each independently 0 or 1;
z is 0, 1, 2, or more; and
WSP is a water soluble polymer, the attachment of which is effected at any reactive moiety in F¹;
and physiologically acceptable salts thereof.

2. The compound of claim 1 wherein at least a or b is 1.

3. The compound of claim 1 wherein b, c, d, e, f, g and h are 0.

4. A compound that binds to an mpl receptor consisting essentially of a structure set out in Formula I,
[(X¹)ₐ-(F¹)_{z}-(X²)_{b}]-(L¹)_{c}-WSP_{d} Formula I
and multimers thereof, wherein:
F¹ is a vehicle;
X¹ is independently selected from:
P¹-(L²)ₑ-
P²-(L³)_{f}-P¹-(L²)ₑ-
P³-(L⁴)_{g}-P²-(L³)_{f}-P¹-(L²)ₑ- and
P⁴-(L⁵)ₕ-P³-(L⁴)_{g}-P²-(L³)_{f}-P¹-(L²)ₑ-
X² is independently selected from:
-(L²)ₑ-P¹,
-(L²)ₑ-P¹-(L³)_{f}P²,
-(L²)ₑ-P¹-(L³)_{f}-P²-(L⁴)_{g}-P³, and
-(L²)ₑP¹-(L³)_{f}-P²-(L⁴)_{g}-P³-(L⁵)ₕ-P⁴
wherein P¹, P², P³, and P⁴ are each independently sequences of pharmacologically active peptides;
L¹, L², L³, L⁴, and L⁵ are each independently linkers;
a, b, c, d, e, f, g, and h are each independently 0 or 1;
z is 0, 1, 2, or more; and
WSP is a water soluble polymer, the attachment of which is effected at any reactive moiety in F¹;
and physiologically acceptable salts thereof.

5. The compound of claim 1, 2, 3, or 4 wherein
F¹ is an Fc domain modified so that it comprises at least one X³ in a loop region;
X³ is independently selected from
-(L⁶)ᵢ-P⁵-(L⁷)ⱼ,
-(L⁶)ᵢ-P⁵-(L⁷)ⱼ-P⁶-(L⁸)ₖ,
-(L⁶)ᵢ-P⁵-(L⁷)ⱼ-P⁶-(L⁸)ₖ-P⁷-(L⁹)ₗ, and
-(L⁶)ᵢ-P⁵-(L⁷)ⱼ-P⁶-(L⁸)ₖ-P⁷-(L⁹)ₗ-P⁸-(L¹⁰)ₘ;
P⁵, P⁶, P⁷, and P⁸ are each independently sequences of pharmacologically active peptides;
L⁶, L⁷, L⁸, L⁹, and L¹⁰ are each independently linkers;
i, j, k, l, and m are each independently 0 or 1; and
z is 1, 2, or more.

6. The compound of claim 5 wherein a and b are each 0.

7. The compound of claim 5 wherein the Fc domain comprises an IgG Fc domain.

8. The compound of claim 7 wherein the Fc domain comprises a sequence selected from SEQ ID NOS: 24 and 25-33.

9. The compound of claim 5 wherein the Fc domain comprises an IgG1 Fc domain.

10. The compound of claim 9 wherein the IgG1 Fc domain comprises SEQ ID NO: 24 and X³ is inserted into or replaces all or part of a sequence selected from SEQ ID NOS: 57, 58, 59, 60, 61, 62, 63, 64, 65, and 66.

11. The compound of claim 10 wherein X³ is inserted into or replaces all or part of a sequence selected from SEQ ID NOS: 67, 68, 69, 70, 71, 72, and 73.

12. The compound of claim 11 wherein X³ is inserted at Leu₁₃₉/Thr₁₄₀.

13. The compound of claim 9 wherein the IgG1 Fc domain comprises SEQ ID NO: 28 and X³ is inserted into or replaces all or part of a sequence selected from SEQ ID NOS: 57, 58, 59, 60, 61, 62, 63, 64, 65, and 66.

14. The compound of claim 13 wherein X³ is inserted at H₅₃/E₅₄, Y₈₁/N₈₂, N₁₁₀/K₁₁₁, L₁₄₃/T₁₄₄, Q₁₇₁/P₁₇₂, E₁₇₃/N₁₇₄, S₁₈₆/D₁₈₇, G₁₈₈/S₁₈₉, or G₂₀₅/N₂₀₆.

15. The compound of claim 9 wherein the IgG1 Fc domain comprises SEQ ID NO: 29 and X³ is inserted into or replaces all or part of a sequence selected from SEQ ID NOS: 57, 58, 59, 60, 61, 62, 64, 65, 66, and 80.

16. The compound of claim 15 wherein X³ is inserted at H₅₃/E₅₄, Y₈₁/N₈₂, N₁₁₀/K₁₁₁, L₁₄₃/T₁₄₄, Q₁₇₁/P₁₇₂, E₁₇₃/N₁₇₄, S₁₈₆/D₁₈₇, G₁₈₈/S₁₈₉, or G₂₀₅/N₂₀₆.

17. The compound of claim 5 wherein the Fc domain comprises an IgG3 Fc domain.

18. The compound of claim 17 wherein the IgG3 Fc domain comprises SEQ ID NO: 30 and X³ is inserted into or replaces all or part of a sequence selected from SEQ ID NOS: 83, 57, 58, 59, 61, 75, 77, 80, 81, and 82.

19. The compound of claim 18 wherein X³ is inserted at H₁₀₀/E₁₀₁, F₁₂₈/N₁₂₉, N₁₅₇/K₁₅₈, M₁₉₀/T₁₉₁, Q₂₁₈/P₂₁₉, E₂₂₀/N₂₂₁, S₂₃₂/D₂₃₃, G₂₃₄/S₂₃₅, or G₂₅₂/N₂₅₃.

20. The compound of claim 5 wherein the Fc domain comprises an IgG2 Fc domain.

21. The compound of claim 20 wherein the Fc domain comprises SEQ ID NO: 31 and X³ is inserted into or replaces all or part of a sequence selected from SEQ ID NOS: 57, 58, 59, 64, 66, 75, 76, 78, 80, and 82.

22. The compound of claim 21 wherein X³ is inserted at H₄₉/E₅₀, F₇₇/N₇₈, N₁₀₆/K₁₀₇, M₁₃₉/T₁₄₀, Q₁₆₇/P₁₆₈, E₁₆₉/N₁₇₀, S₁₈₁/D₁₈₂, G₁₈₃/S₁₈₄, or G₂₀₁/N₂₀₂.

23. The compound of claim 5 wherein the Fc domain comprises an IgG4 Fc domain.

24. The compound of claim 23 wherein the Fc domain comprises SEQ ID NO: 32 and X³ is inserted into or replaces all or part of a sequence selected from SEQ ID NOS: 84, 57, 59, 61, 64, 65, 74, 75, 79, and 80.

25. The compound of claim 24 wherein X³ is inserted at Q₅₀/E₅₁, F₇₈/N₇₉, N₁₀₇/K₁₀₈, M₁₄₀/T₁₄₁, Q₁₆₈/P₁₆₉, E₁₇₀/N₁₇₁, S₁₈₂/D₁₈₃, G₁₈₄/S₁₈₅, or G₂₀₂/N₂₀₃.

26. The compound of claim 5 wherein the Fc domain comprises SEQ ID NO: 33 and X³ is inserted into or replaces all or part of a sequence selected from SEQ ID NOS: 57, 58, 62, 59, 61, 64, 66, 75, 80, and 82.

27. The compound of claim 26 wherein X³ is inserted at H₁₁₂/E₁₁₃, F₁₄₀/N₁₄₁, N₁₆₉/K₁₇₀, M₂₀₄/T₂₀₅, Q₂₃₂/P₂₃₃, E₂₃₄/N₂₃₅, S₂₄₆/D₂₄₇, G₂₄₈/S₂₄₉, or G₂₆₈/N₂₆₉.

28. The compound of any of claims 1-27, wherein P is independently selected from the group consisting of:
(SEQ ID NO: 6)
QGCSSGGPTLREWQQCRRAQHS;
(SEQ ID NO: 11)
QGCSSGGPTLREWQQCVQAQHS (FcL2);
(SEQ ID NO: 12)
QGCSSGGPTLREWQQCVGAQHS (FcL3);
(SEQ ID NO: 13)
QGCSSGGPTLREWQQCVHAQHS (FcL4);
(SEQ ID NO: 14)
QGCSSGGPTLREWQQCQGAQHS (FcL5);
(SEQ ID NO: 15)
QGCSSGGPTLREWQQCVRPQHS (FcL6);
(SEQ ID NO: 16)
QGCSSGGPTLREWQQCFRPQHS (FcL7);
(SEQ ID NO: 17)
QGCSSGGPTLEEWQQCFKAQHS (FcL8);
(SEQ ID NO: 18)
QGCSSGGPTLREWQQCVKPQHS (FcL9);
(SEQ ID NO: 19)
QGCSSGGPTLREWQQCVRAQHS (FcL10);
(SEQ ID NO: 20)
QGCSSGGPTLREWQQCRPAQHS (FcL11);
(SEQ ID NO: 21)
QGCSSGGPTLREWQQCRRPQHS (FcL12);
(SEQ ID NO: 22)
QGCSSGGPTLREWQQCQRAQHS (FcL13);
and
(SEQ ID NO: 23)
QGCSSGGPTLREWQQCSRAQHS (FcL14).

29. A polynucleotide that encodes a compound of any of claims 1-28.

30. A vector that comprises the polynucleotide of claim 29.

31. A host cell that comprises the vector of claim 30.

32. A method of producing a compound that binds to an mpl receptor which comprises growing the host cell of claim 31 in a suitable nutrient medium and isolating said compound from said cell or nutrient medium.
